(19) United States
(12) Patent Application Publication
(10) Pub. No.: US 2017/0079655 A1
(43) Pub. Date: Mar. 23, 2017

(54) TISSUE GATHERING BASKET ENHANCEMENTS

(71) Applicant: Empire Technology Development LLC, Wilmington, DE (US)

(72) Inventors: Worth Walters, Los Gatos, CA (US); Todd Dickson, Santa Clara, CA (US); Abby George, Mountain View, CA (US); Brian Moore, Sunnyvale, CA (US); Sam Malenowski, San Francisco, CA (US)

(21) Appl. No.: 14/858,970

(22) Filed: Sep. 18, 2015

(51) Int. Cl. A61B 17/10; A61B 17/221; A61B 17/22; A61B 17/08; A61B 17/00

(52) U.S. Cl. CPC A61B 17/10; A61B 17/08; A61B 17/0057; A61B 17/22031; A61B 17/221; A61B 2017/081; A61B 2017/00818; A61B 2017/00641; A61B 2017/00632; A61B 2017/00862; A61B 2017/2215

(57) ABSTRACT

Several embodiments disclosed herein relate to apparatus and methods for tissue gathering. In some examples, disclosed is a tissue gathering device that includes a basket and a cylindrical shaft. The basket can include a plurality of tines and the cylindrical shaft can include a lumen that is disposed about a portion of the proximal end of the basket. In some examples, the basket can include 4, 6, 7, or 8 tines. The tines of the basket can have a plurality of different configurations.

### BACKGROUND

An outpouching of the colon or other body lumen, called a diverticulum, can become the site for inflammation known as diverticulitis, microperforation and/or bleeding. Current treatments may involve the surgical removal of segments of the body lumen. For extreme cases of diverticulitis, treatment can involve colon resection and placement of a colostomy. This approach results in significant healthcare costs and substantial pain for patients.

### SUMMARY

Disclosed is a tissue gathering device. In some embodiments, the tissue gather device includes a basket with a plurality of tines and a cylindrical shaft with a lumen that can be disposed about a portion of the proximal end of the basket. The basket can be made of a material selected from an elastic polymer, such as polyurethane, polyethylene terephthalate, or polyethyleneoxide, or a metal, such as nitinol or cobalt-chromium,

In other embodiments, the tissue gathering device can include a basket with at least about 4 tines. In other embodiments, the basket can include at least 6 tines. In other embodiments, the basket can include at least 7 tines. In other embodiments, the basket can include at least 8 tines.

In some embodiments, the tissue gathering device includes a basket with a plurality of tines of different lengths.

In some embodiments, the tissue gathering device includes a basket with a plurality of tines that form an oval when the plurality of tines of the basket is depressed onto a surface.

In some embodiments, the tissue gathering device includes a plurality of tines that include a first bend and a second bend, wherein the first bend extends the tine radially outward, and the second bend extends the tine radially inward along the length of the tine. In other embodiments, the second bend can have an angle between 0-20 degrees, 20-40 degrees, 40-60 degrees, 60-80 degrees, 80-100 degrees, 100-120 degrees, 120-140 degrees, 140-160 degrees, 160-170 degrees, less than 180 degrees.

In some embodiments, the tissue gathering device includes a plurality of tines with a diameter that widens along the length of each of the plurality of tines. In some embodiments, each of the plurality of tines has a distal end with a recessed arch such that the distal end of each of the plurality of tines is configured to form a pair of spikes. In some embodiments, the distal end of each of the plurality of tines has more than one recessed arch and forms a plurality of spikes.

In some embodiments, the tissue gathering device includes a plurality of tines with a diameter that widens along the length of each of the plurality of tines and ends in a wide base. In some embodiments, each of the plurality of tines has a narrow protrusion extending from the wide base. In some embodiments, the distal end of each of the plurality of tines has more than one narrow protrusion.

In some embodiments, the tissue gathering device includes a plurality of tines with a variable diameter along the length of each of the plurality of tines that decreases and then increases so as to form a narrowed waist in each of the plurality of tines.

In some embodiments, the tissue gathering device includes a plurality of tines further comprises a shape element attached to the proximal end. In some embodiments, the shape element is a bump or a protrusion.

In some embodiments, the tissue gathering device includes a rotation mechanism, wherein the rotation mechanism is attached to the proximal end of the basket to allow rotation of the basket and the plurality of tines. In some embodiments, the rotation mechanism is configured to rotate the basket and the plurality of tines along an arc. In some embodiments, the rotation can be between 0-20 degrees, 20-40 degrees, 40-60 degrees. In some embodiments the rotation mechanism is a trigger, a dial, or a level.

In some embodiments, the tissue gathering device includes a plurality of tines that form an oval.

Disclosed is also a method for gathering tissue in treating a diverticulum. In some embodiments the method includes positioning a distal end of a device along an outer wall of a colon at a diverticulum, wherein the device comprises a basket, a cylindrical shaft, and a pushing apparatus disposed coaxially between the basket and the cylindrical shaft, and wherein the distal end of the cylindrical shaft is disposed about the proximal end of the basket, and wherein the basket comprises a plurality of tines. In some embodiments, the method can further include inverting the diverticulum by inserting the distal end of the pushing apparatus into the inverted diverticulum. In some embodiments, the method can further include aligning the basket of the device with the inverted diverticulum such that the plurality of tines of the basket is disposed about the rim of the inverted diverticulum. In some embodiments, the method can further include moving the cylindrical shaft in a proximal direction to retract the basket within the distal end of the cylindrical shaft. In some embodiments, the method can further include engaging the plurality of tines of the basket with the tissue around the inverted diverticulum.

In some embodiments, the method includes a basket where each of the tines has a first bend and second bend, and wherein the first bend extends the tine radially outward and the second bend extends the tine radially inward along the length of the tine. In other embodiments, the method can further include the step of moving the cylindrical shaft in a distal direction such that the length of the basket between the first bend and the second bend is retracted into the distal end of the cylindrical shaft such that the angle of the second bend gathers the tissue around the inverted diverticulum.

In some embodiments, the method includes a basket where each of the tines has a diameter that widens along the length of each of the plurality of tines and has a distal end with a recessed arch such that the distal end of each of the plurality of tines is configured to form a pair of spikes. In other embodiments, the method includes a basket that includes the step of engaging the spikes and recessed arch of the distal end of the plurality of tines with the tissue around the inverted diverticulum.

In some embodiments, the method includes a basket where each of the tines has a diameter that widens along the length of each of the plurality of tines and ends in a wide base, and each of the plurality of tines has a narrow protrusion extending from the wide base. In other embodiments, the method further includes puncturing the tissue around the inverted diverticulum with the narrow protrusion from each of the plurality of tines, wherein the wide base limits the puncturing of the tissue by the narrow protrusion and aids in securing tissue about the diverticulum as the cylindrical shaft is moved in a distal direction.

In some embodiments, the method includes a basket where each of the plurality of tines has a variable diameter along the length of each of the plurality of tines that decreases and then increases so as to form a narrowed waist in each of the plurality of tines. In other embodiments, the method further includes moving the cylindrical shaft in a proximal direction allows the narrowed waist of each of the plurality of tines to gather and secure the tissue of the inverted diverticulum between each of the plurality of tines. In other embodiments, the method further includes a rotation mechanism configured to interact with the cylindrical shaft and the basket such that moving the cylindrical shaft in a proximal direction rotates the basket and the plurality of tines.

In some embodiments, the method has a device that further includes a clip comprising a plurality of clip tines configured to engage with each of the plurality of tines of the basket, and wherein the method further comprises engaging the plurality of tines with the plurality of clip tines of the clip such that the tissue about the diverticulum is secured between the plurality of tines and the plurality of clip tines.

Disclosed is also a system of gathering tissue in treating a diverticulum. In some embodiments, the system includes a device comprising a basket including a plurality of tines, a cylindrical shaft including a lumen, wherein the cylindrical shaft is disposed about a portion of the proximal end of the basket and wherein the lumen has a circular cross-section, and a clip including a plurality of clip tines configured to engage with each of the plurality of tines of the basket.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings.

FIG. 1A illustrates a side view of an embodiment of a device for inverting diverticulum with all of the components of the device exposed.

FIG. 1B illustrates a side view of the device illustrated in FIG. 1A with a few of the components retracted.

FIGS. 1C-K illustrate a plurality of views of a method for treating diverticulum disease using the device of FIG. 1A.

FIG. 1L illustrates a flowchart of an embodiment of the method for treating diverticulum disease illustrated in FIGS. 1C-K.

FIG. 2A illustrates a side view of another embodiment of a device for inverting diverticulum.

FIGS. 2B-H illustrate a plurality of views of a method for treating diverticulum disease using the device of FIG. 2A.

FIG. 3A illustrates a side view of another embodiment of a device for inverting diverticulum.

FIG. 3B illustrates a top view of an embodiment of a closure clip in a relaxed configuration wherein the closure clip can be used with the devices illustrated in FIGS. 1A, 2A, and 3A.

FIG. 3C illustrates a side view of the closure clip of FIG. 3B when it is loaded on any of the devices illustrated in FIGS. 1A, 2A, and 3A.

FIG. 3D illustrates a side view of the closure clip of FIG. 3B as it is delivered to a target site.

FIG. 3E illustrates a side view of an embodiment of a clip tube of the device of FIG. 3A.

FIG. 3F illustrates the clip tube of FIG. 3E that is loaded with the closure clip illustrated in FIG. 3D.

FIGS. 3G-I illustrate a plurality of views of the distal end of an embodiment of a push rod of the device of FIG. 3A.

FIG. 3J illustrates a side view of the clip tube of FIG. 3E and push rod of FIGS. 3G-I that is loaded with the closure clip illustrated in FIG. 3D.

FIG. 3K illustrates a side view of the apparatus of 3J where the closure clip is configured for delivery as illustrated in FIG. 3E.

FIG. 3L illustrates an embodiment of a sheath of the device of FIG. 3A.

FIGS. 3M-S illustrate a plurality of views of a method for treating diverticulum using the device of FIG. 3A.

FIGS. 4A-B are side views of dimensional presentations of diverticula and their inverted counterparts.

FIGS. 5A-E are perspective and side views of one embodiment of a gathering element with tines of different lengths used in a method for treating diverticulum disease.

FIGS. 6A-C are side views of one embodiment of a gathering element with tines with inward pointed tine ends used in a method for treating diverticulum disease.

FIGS. 7A-E are perspective view of a plurality of gathering elements used in a method for treating diverticulum disease.

FIGS. 8A-B are views of the embodiment of a gathering element with double pointed tip tines of FIG. 7C used in the method for treating diverticulum disease.

FIGS. 8C-D are views of the embodiment of a gathering element with a sharp spike distal end of FIG. 7A used in a method for treating diverticulum disease.

FIGS. 9A-C are side and perspective views of one embodiment of a gathering element with narrowed tine ends used in a method for treating diverticulum disease.

FIGS. 10A-D are views of one embodiment of a gathering element with twisting basket used in a method for treating diverticulum disease.

FIGS. 11A-D are distal views of a gathering element with different numbers of tines used in a method for treating diverticulum disease.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

Disclosed herein are methods and devices for treating diverticular disease. The method can include positioning a distal end of a tissue gathering device along an outer wall of a colon at a diverticulum, inverting the diverticulum, aligning the basket of the device with the inverted diverticulum, moving the cylindrical shaft in a proximal direction to retract the basket, and engaging the plurality of tines of the basket with the tissue around the inverted diverticulum. The device can include a basket including a plurality of tines and a cylindrical shaft including a lumen, wherein the cylindrical shaft is disposed about a portion of the proximal end of the basket.

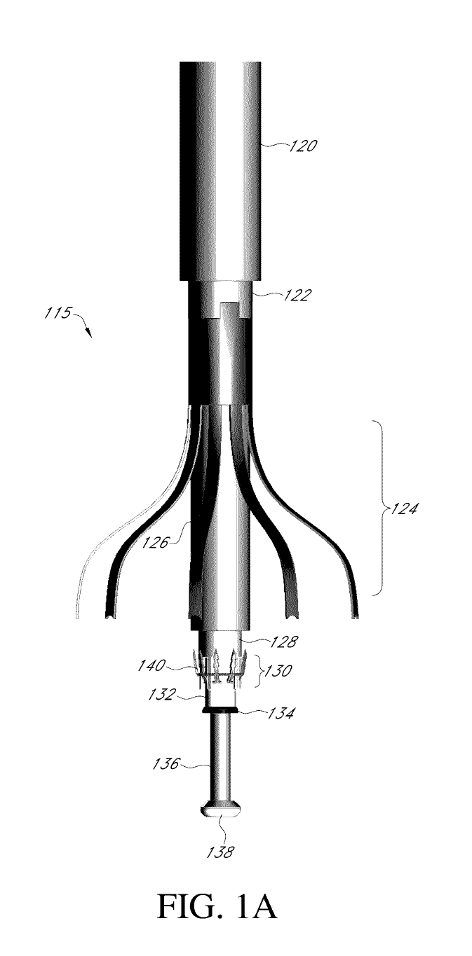

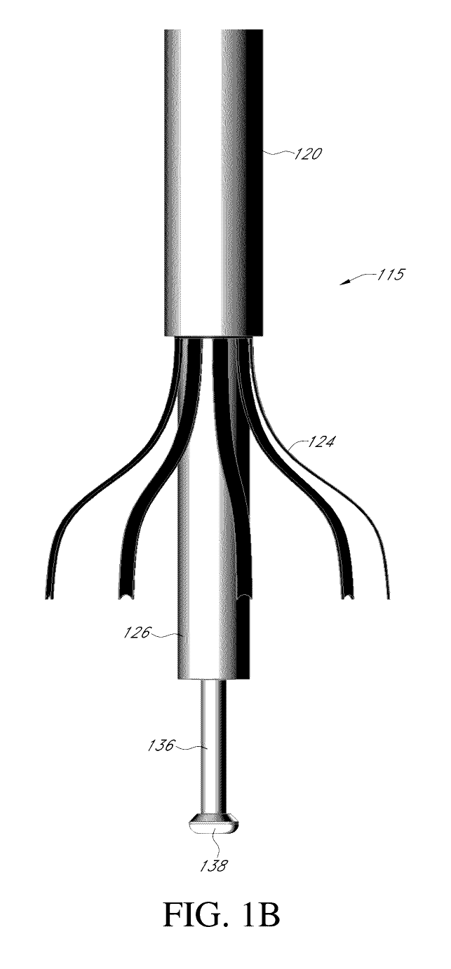

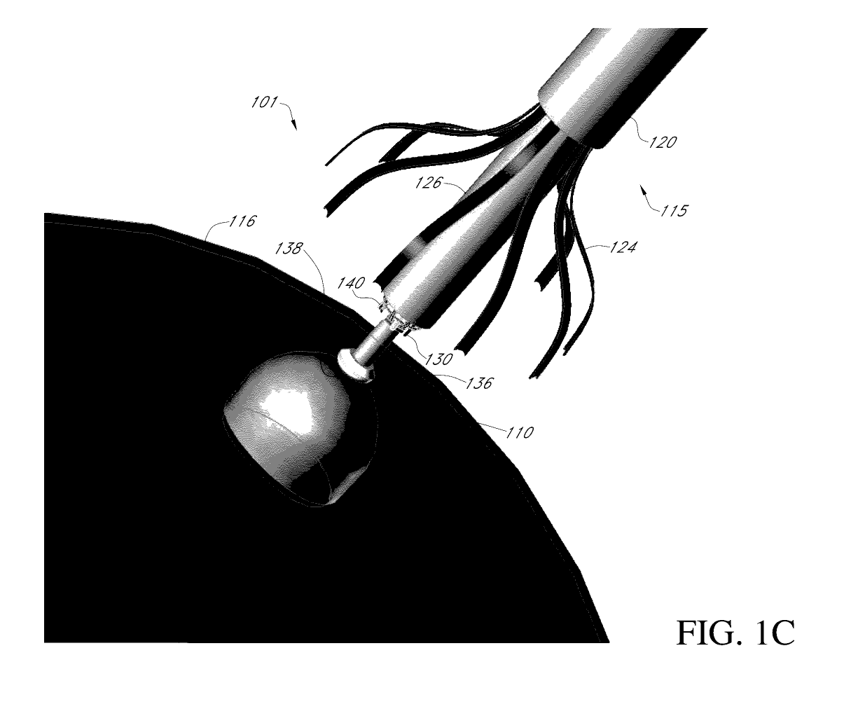

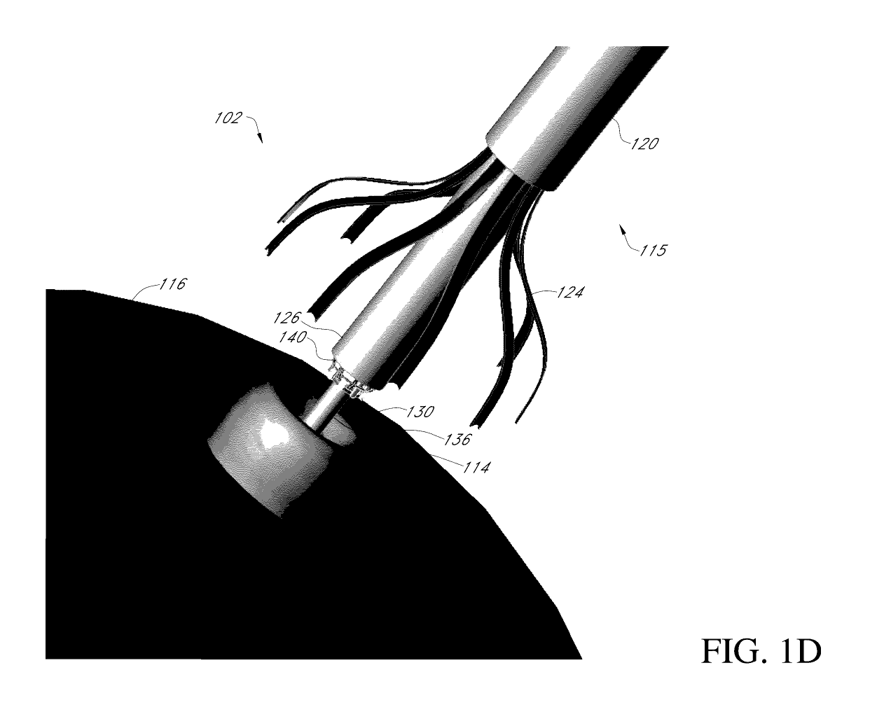

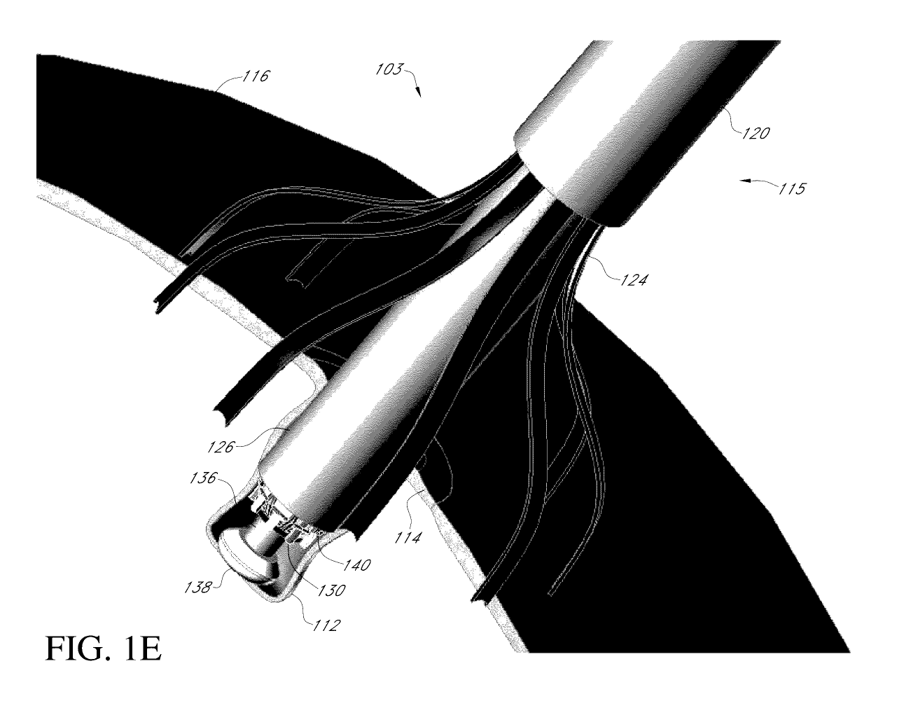

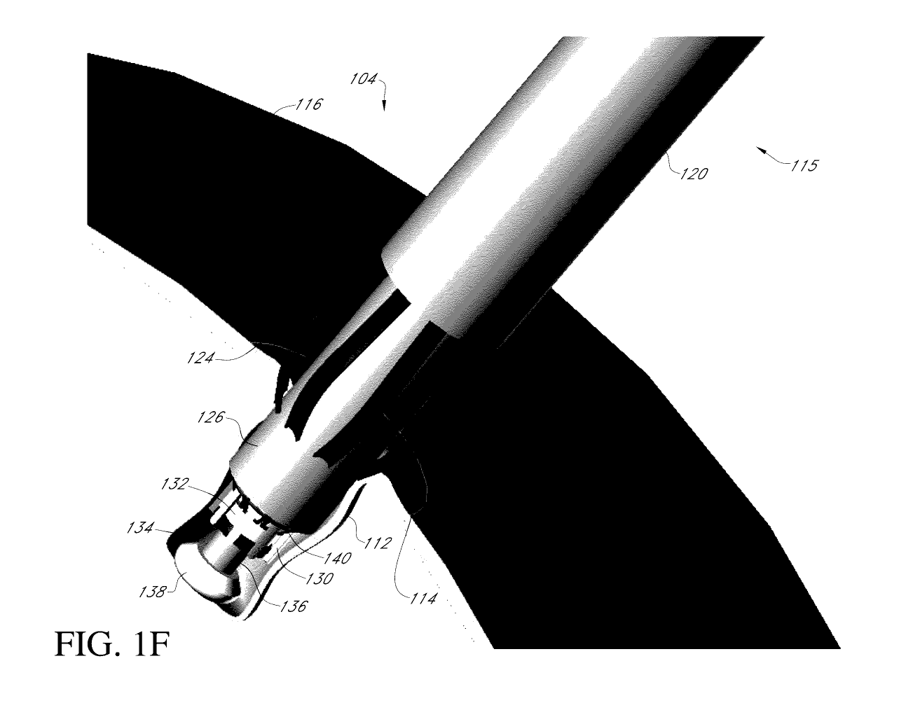

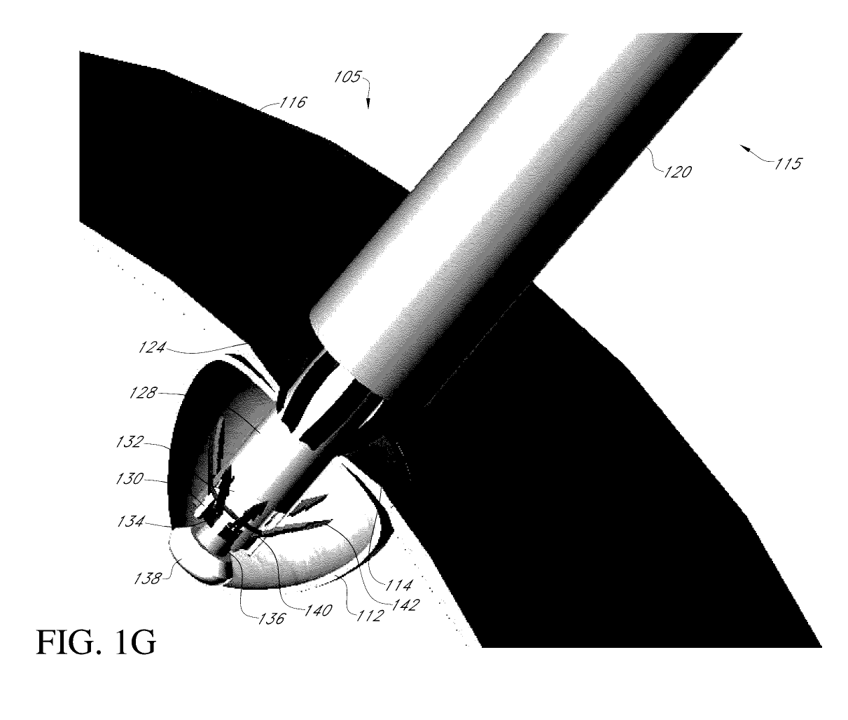

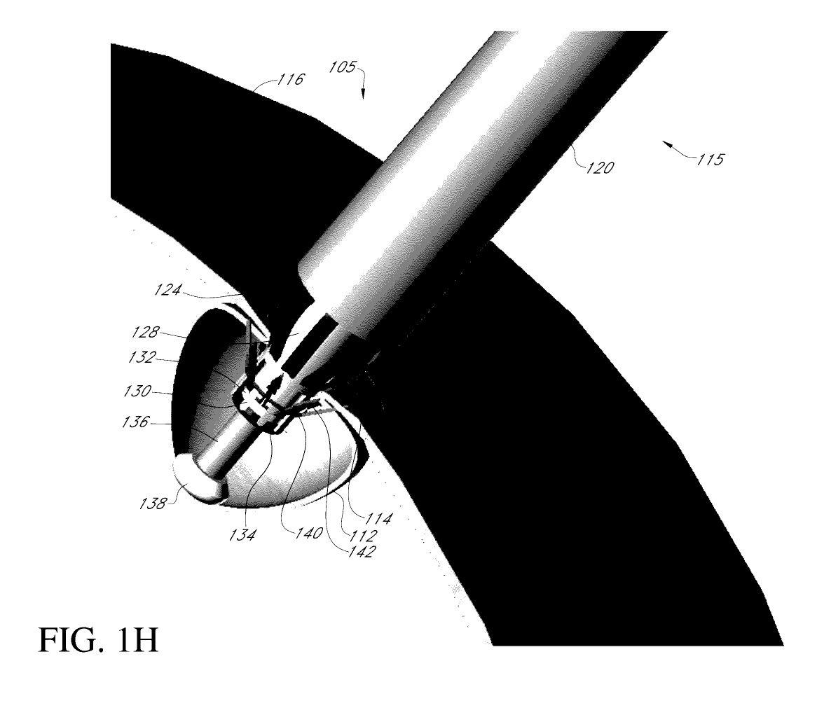

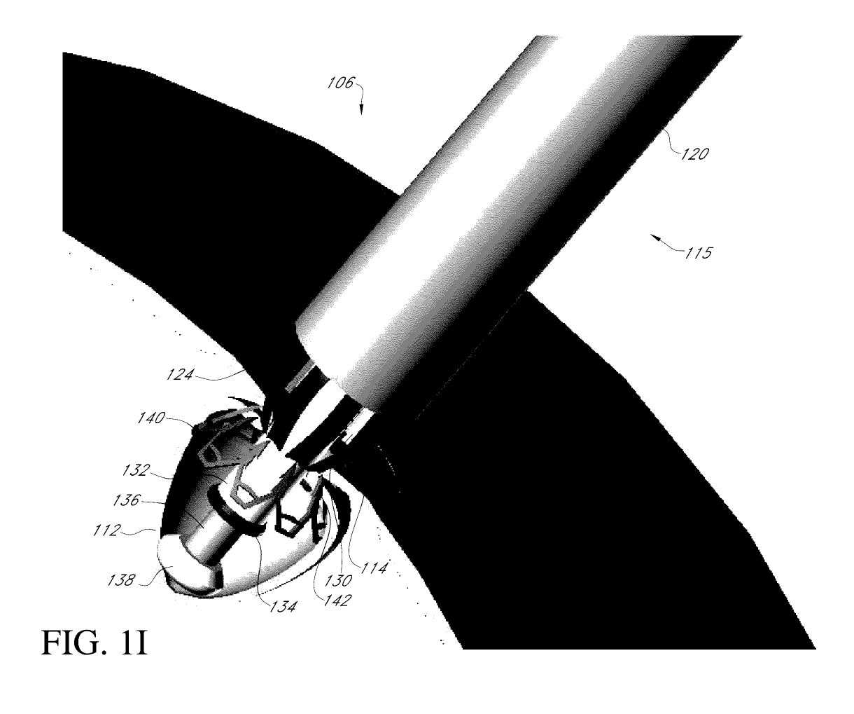

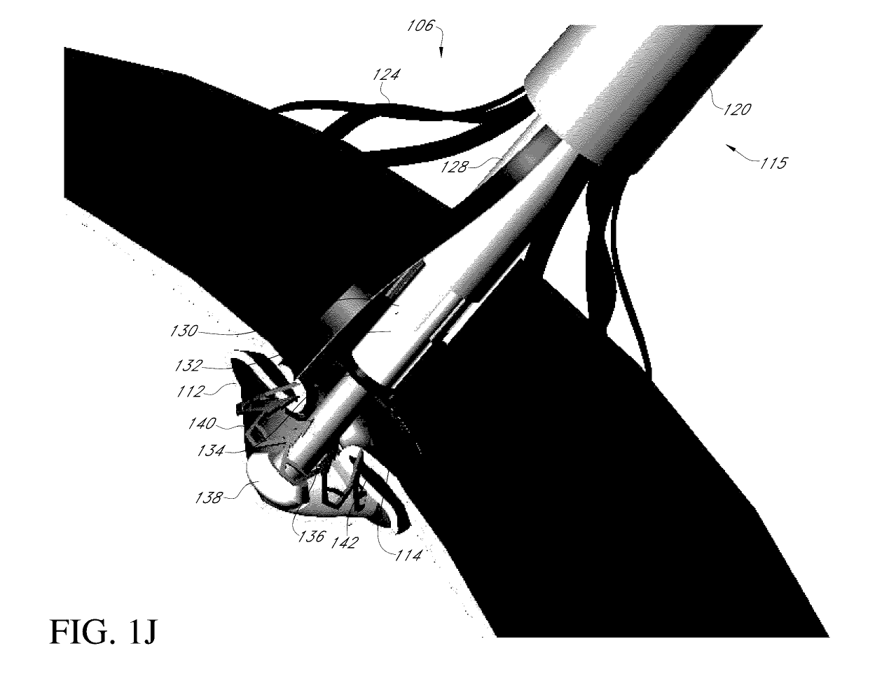

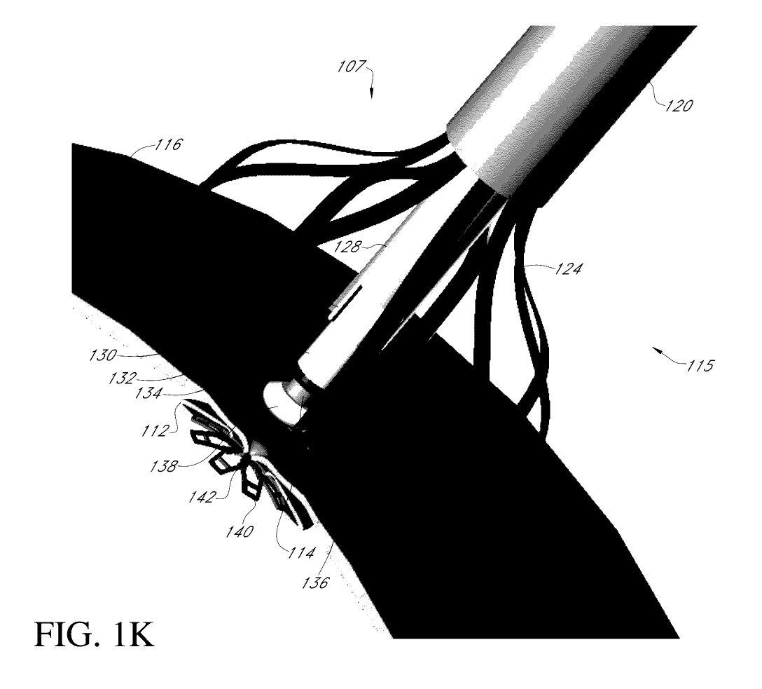

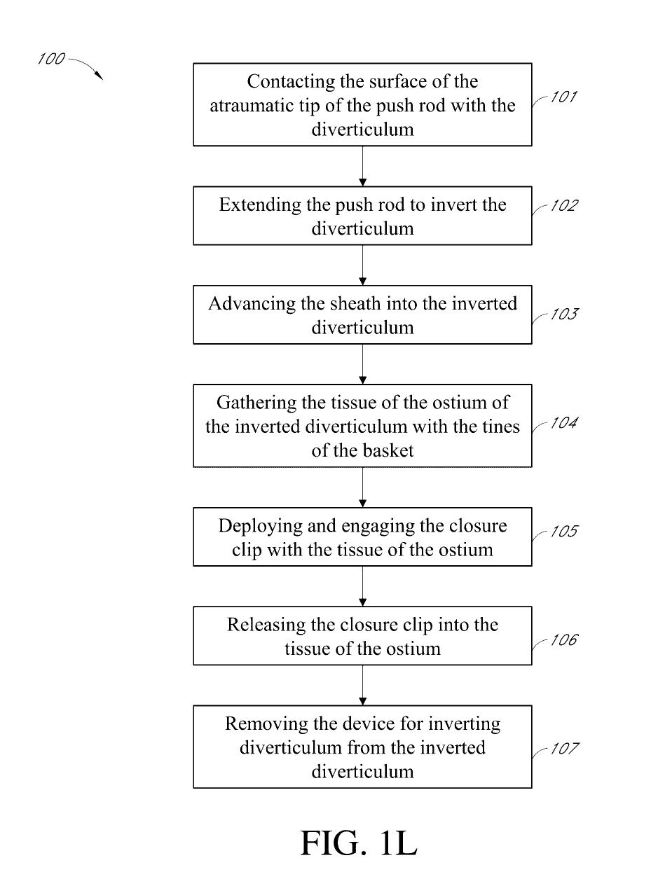

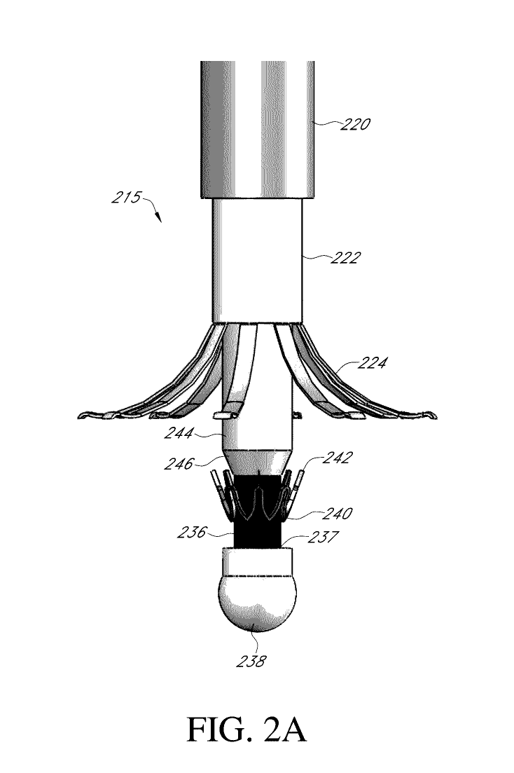

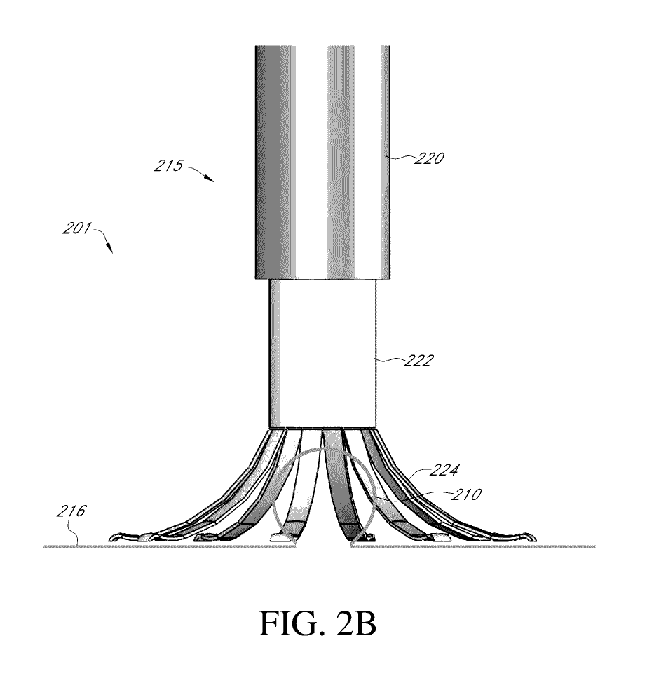

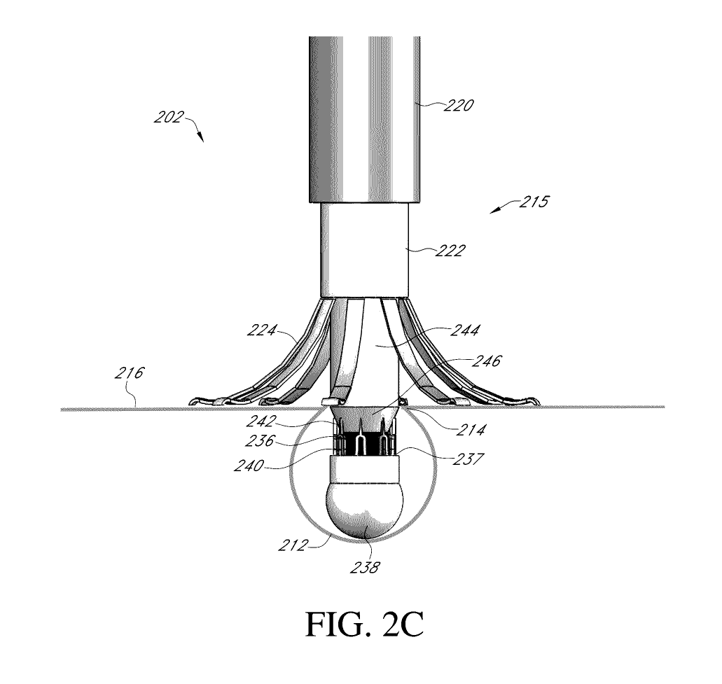

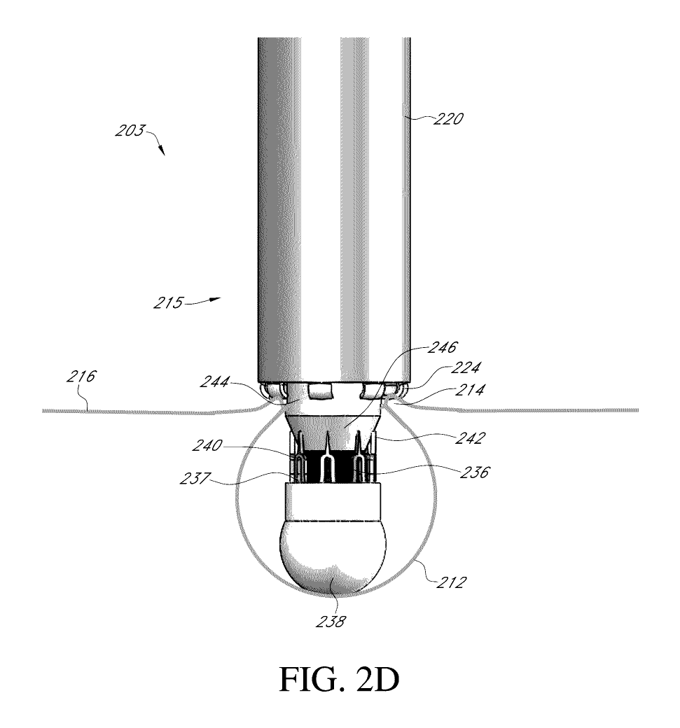

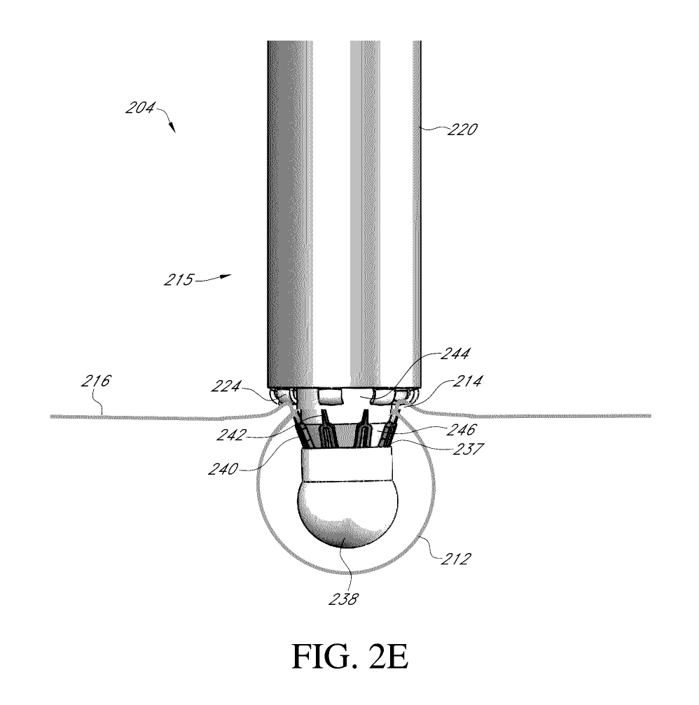

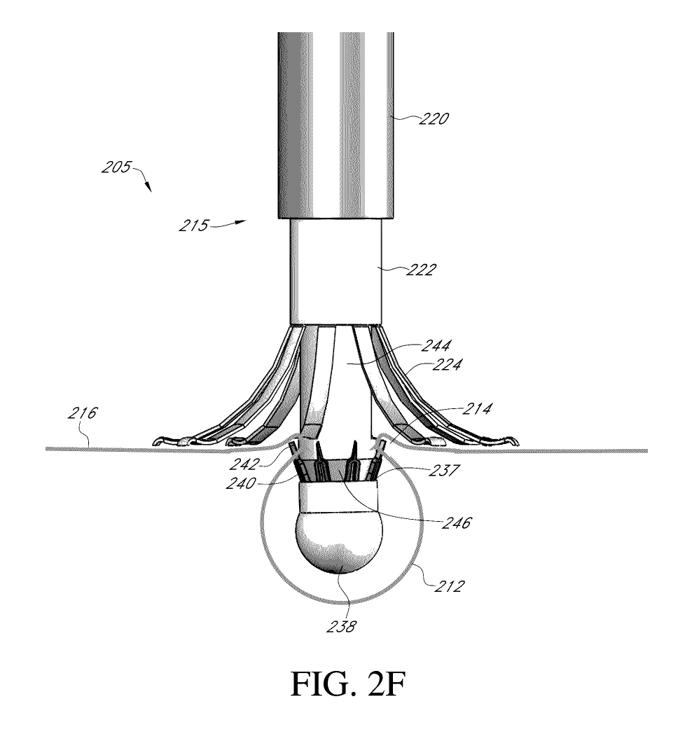

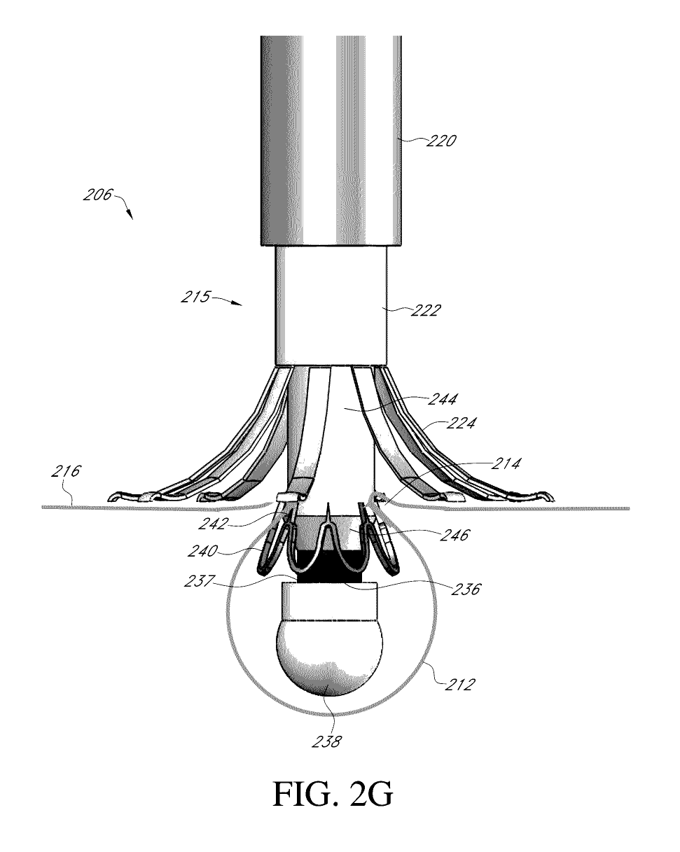

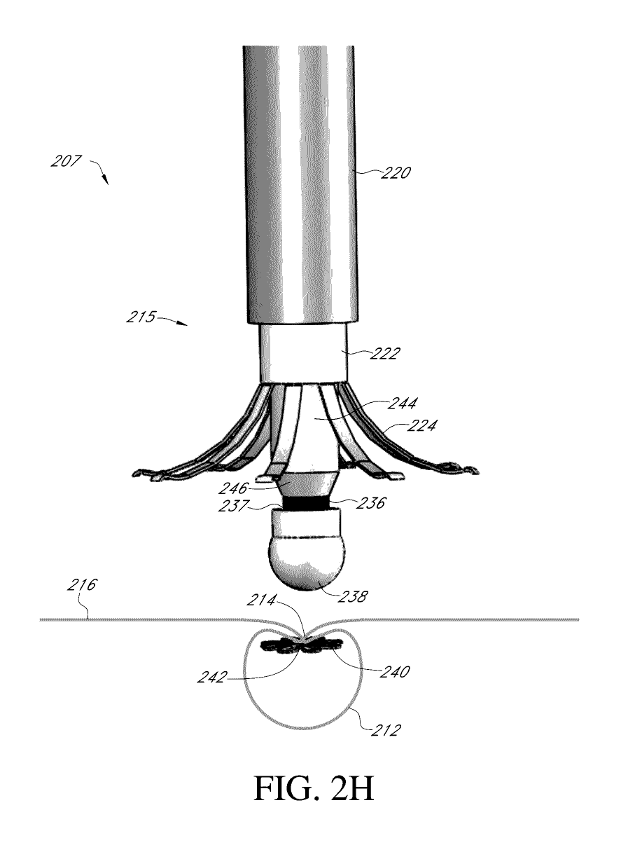

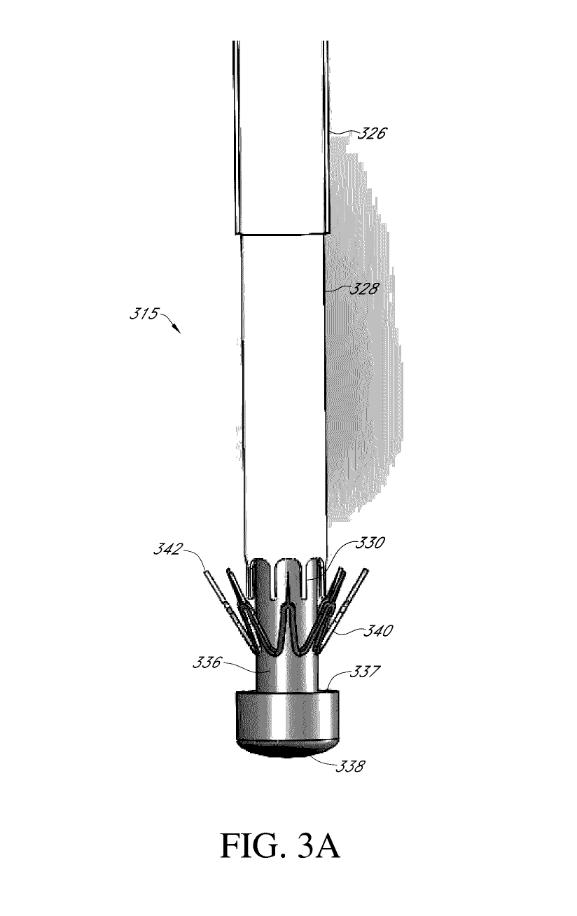

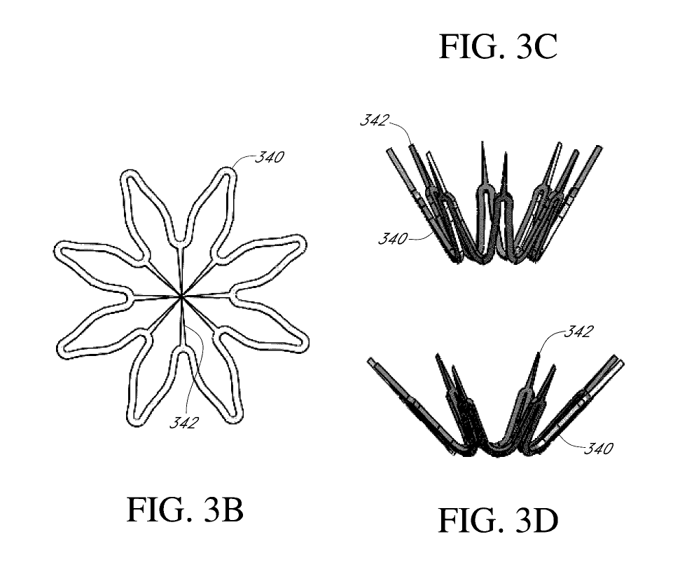

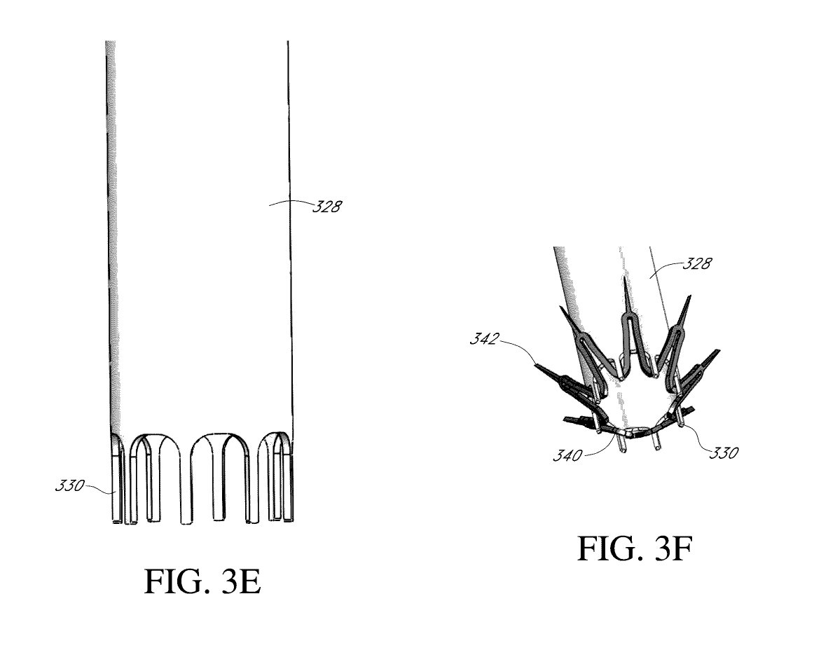

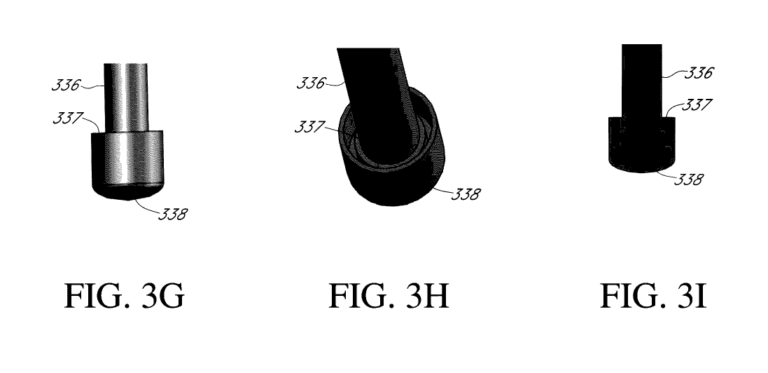

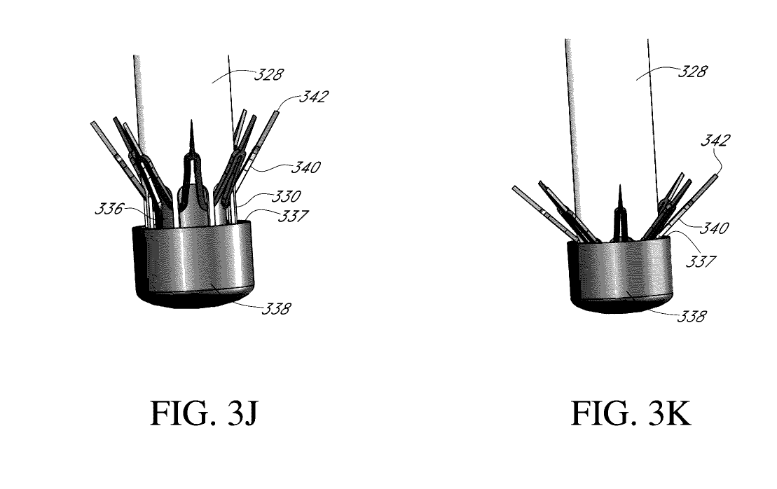

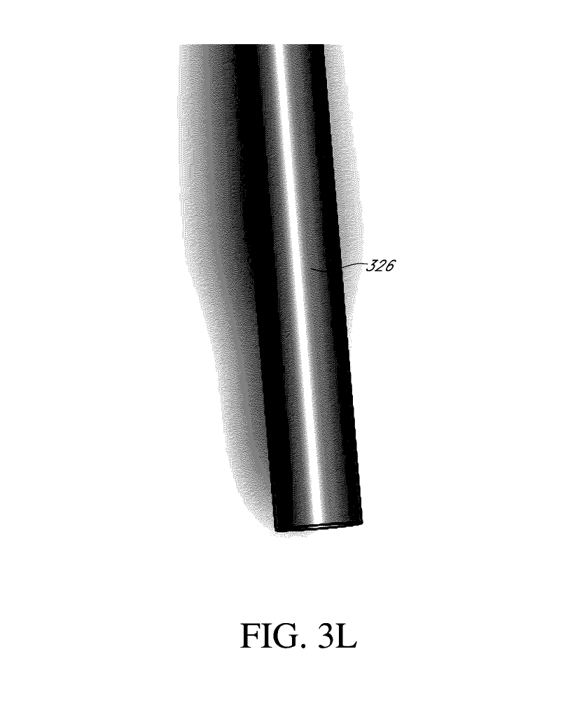

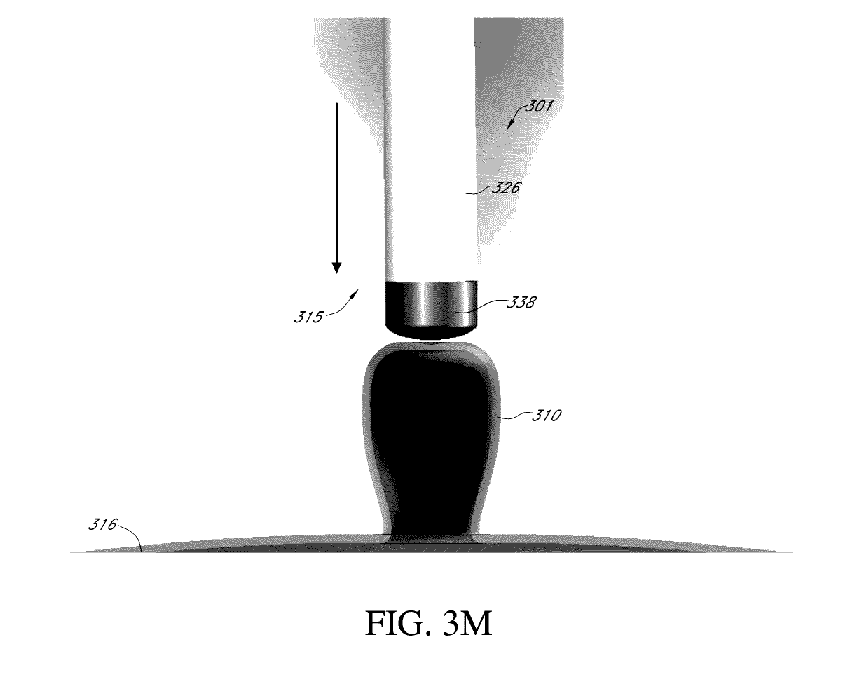

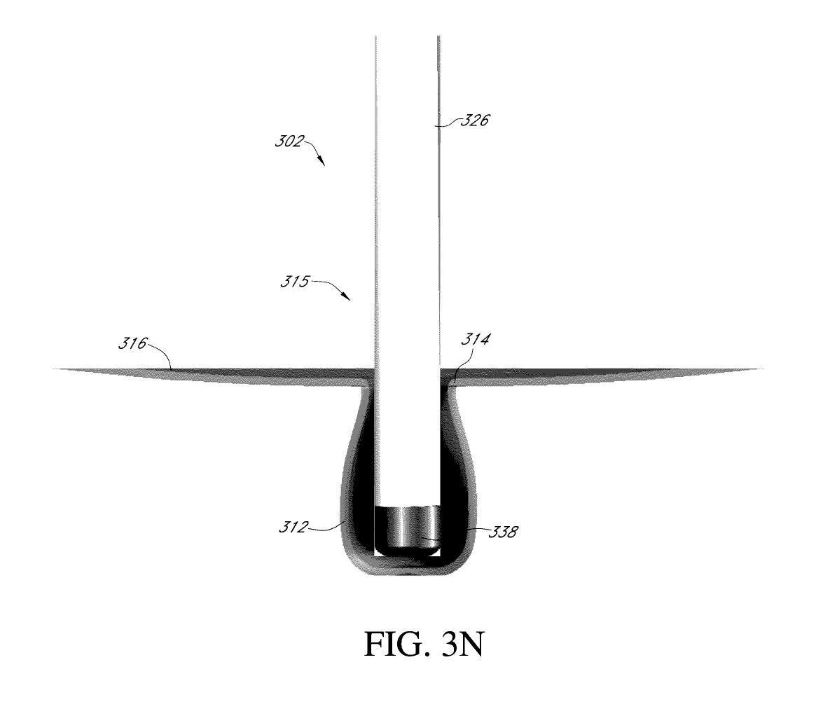

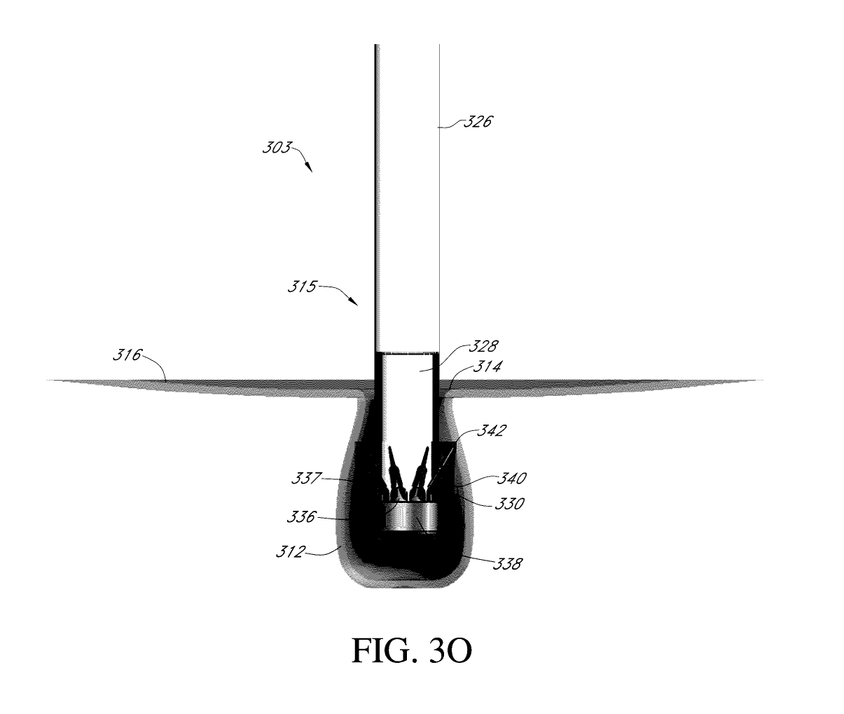

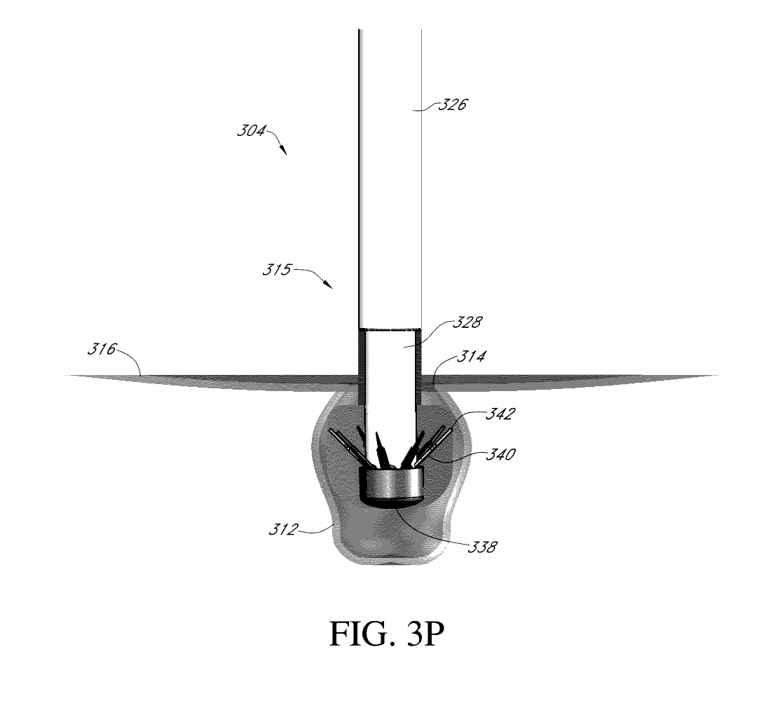

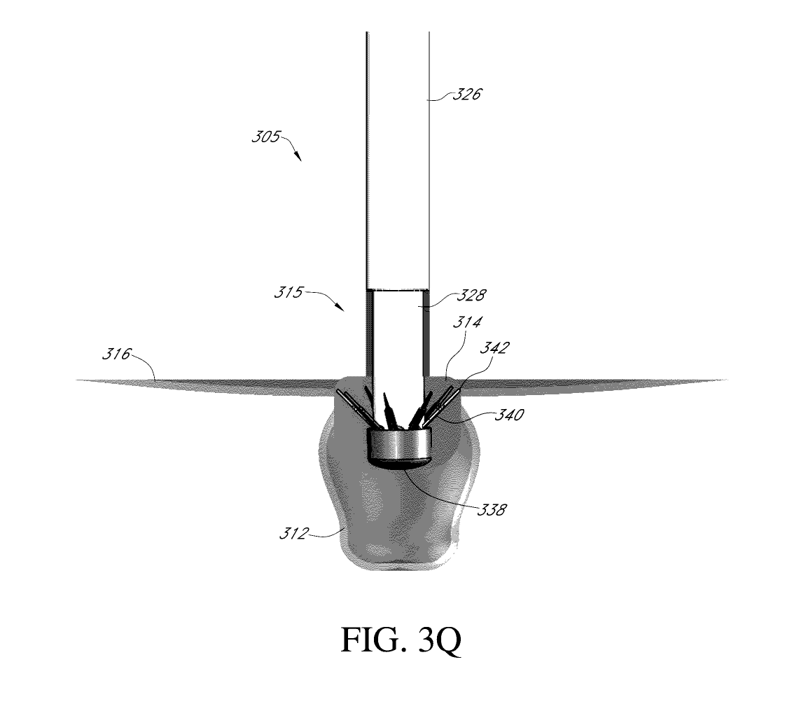

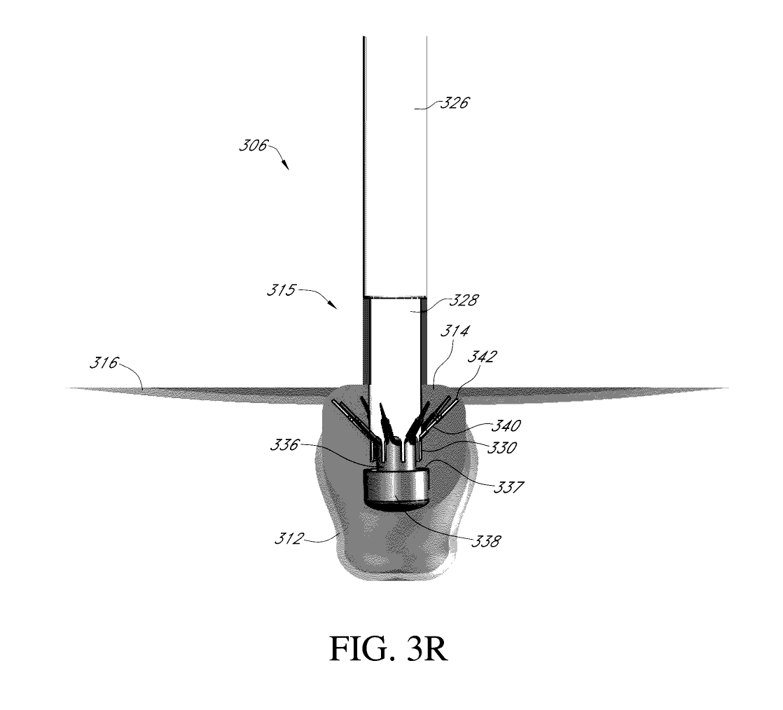

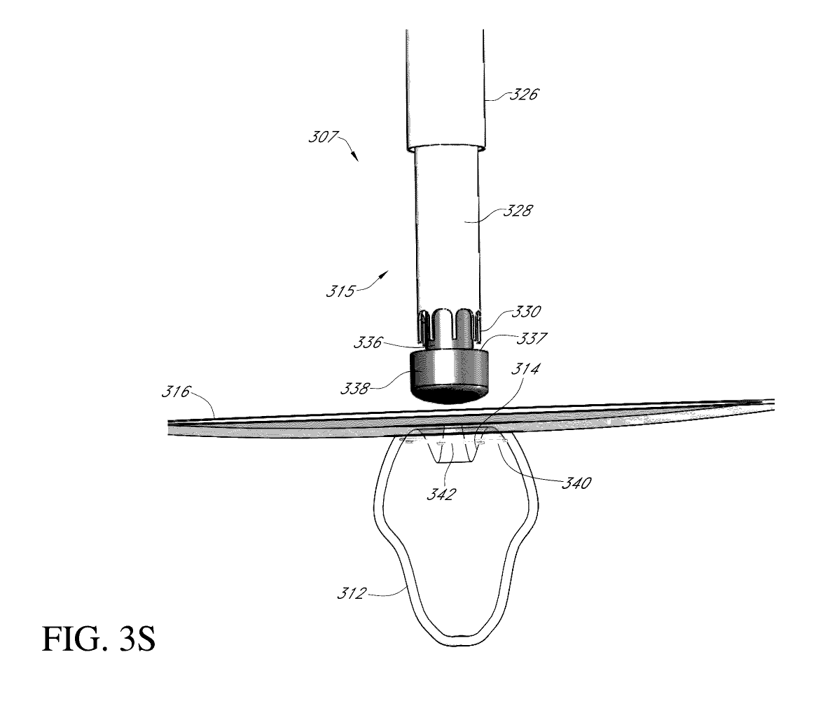

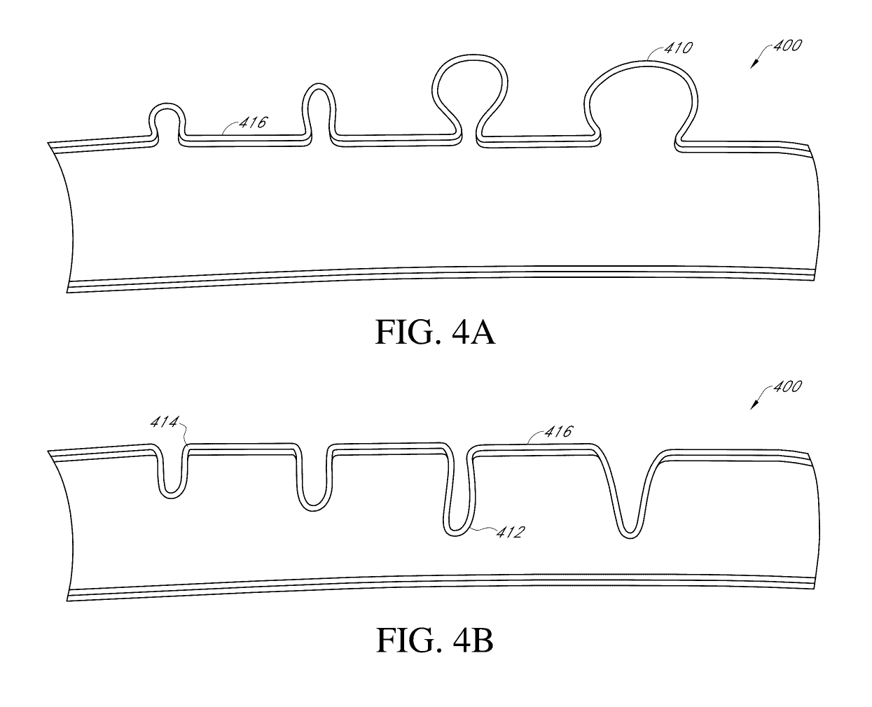

FIGS. 1A-G, 2A-H, and 3A-R illustrate a plurality of views of a device for inverting diverticulum in a method for treating diverticulum disease. FIGS. 1A-G illustrate a plurality of views of embodiments of a device for inverting diverticulum 115 in an embodiment of a method for inverting diverticula 100. FIGS. 2A-H illustrate a plurality of views of another embodiment of a device for inverting diverticulum 215 in some embodiments of a method of inverting diverticula. Finally, FIGS. 3A-R illustrate a plurality of view of another embodiment of a device for inverting diverticulum 315 in some embodiments of a method of inverting diverticula.

Turning first to the embodiment of a device for inverting diverticulum 115 illustrated in FIG. 1A, in some embodiments the device for inverting diverticulum 115 is composed of a plurality of components that are disposed coaxially about each other. As will be illustrated in the method of inverting diverticula 100, each of the components of the device for inverting diverticulum 115 are configured to be moveable relative to each other.

In some embodiments the device for inverting diverticulum 115 can include a closer 120, a basket shaft 122, a sheath 126, a clip tube 128, a tube stop 132, and a push rod 136. In some embodiments, the basket shaft 122 can further include a basket 124 at the distal end. As well, in some examples, the clip tube 128 can include a section including locking structures 130 at the distal end. As will be discussed, the locking structures 130 of the clip tube 128 can help to retain a closure clip 140. In some embodiments, the push rod 136 can further include an atraumatic tip 138 at the distal end. Each of the tubular components of the device for inverting diverticulum 115 can be composed of hardened steel.

As seen in FIG. 1A, the push rod 136 is located at the center of the device for inverting diverticulum 115. In some embodiments, the push rod 136 includes an atraumatic tip 138 at the distal end. As will be discussed below, the atraumatic tip 138 of the push rod 136 can serve to invert a target diverticulum during the method of inverting diverticula 100.

In some examples, a tube stop 132 is disposed coaxially about the push rod 136. In some variants, a flange 134 is located at the distal end of the tube stop 132 such that the flange 134 forms a wider diameter than the distal opening of the tube stop 132. The push rod 136 and the tube stop 132 can move relative to each other such that the push rod 136 can be withdrawn into the tube stop 132. In some examples, the atraumatic tip 138 has a sufficiently wide diameter such that it prevents the push rod 136 from being withdrawn entirely into the tube stop 132. As will be discussed in more detail below, the tube stop 132—in particular the flange 134 of the tube stop 132—can help to adjust the angle on which the closure clip 140 is retained on the clip tube 128 as well as adjust the angle that the closure clip 140 penetrates into the target diverticulum.

In some variants, a clip tube 128 is disposed about the tube stop 132. As noted above, in some examples, the clip tube 128 includes a plurality of locking structures 130 that are located at the distal end of the clip tube 128. The locking structures 130 can be configured to retain a closure clip 140 on the distal end of the device for inverting diverticulum 115. The clip tube 128 is configured such that it is moveable relative to the tube stop 132. In some examples, the locking structures 130 of the clip tube 128 can extend past the flange 134 of the tube stop 132. As will be discussed below, the locking structures 130 can help to retain the closure clip 140 on the device for inverting diverticulum 115. In some variants, along with the flange 134, the locking structures 130 can adjust the angle of the closure clip 140 on the distal end of the device for inverting diverticulum 115 to better allow the closure clip 140 to engage with the tissue of the target inverted diverticulum. As well, the interaction of the flange 134 and the locking structures 130 can also allow the closure clip 140 to be deployed into the inverted diverticulum.

In some embodiments, a sheath 126 can be disposed about the clip tube 128. In some examples, the diameter of the sheath 126 should be wide enough such that it can accommodate the clip tube 128 and the attached closure clip 140. In some examples, the purpose of the sheath 126 is to prevent the closure clip 140 or the locking structures 130 of the clip tube 128 from catching onto anything prior to deployment or placed into the inverted diverticulum.

In some examples, the device for inverting diverticulum 115 can further include a basket shaft 122. As illustrated in FIG. 1A, in some variants, the basket shaft 122 can include a basket 124 located at the distal end of the basket shaft 122. As the basket shaft 122 is configured to be moveable relative to the sheath 126, the basket shaft 122 can be extended or withdrawn proximal and/or distal to the other components of the device for inverting diverticulum 115. As will be described in more detail below, the basket 124 is configured to retain the tissue about the ostium of the inverted diverticulum. This can help to better deploy the closure clip 140 into the target tissue.

Lastly, in some embodiments, the device for inverting diverticulum 115 can further include a closer 120. In some examples, the closer 120 is configured to be disposed about the internal components of the device for inverting diverticulum 115. As the closer 120 is moveable relative to the basket shaft 122, the closer 120 can extend distally to cover and retain the basket 124 of the basket shaft 122. In some examples this can help to maintain the minimal profile of the device for inverting diverticulum 115 prior to use of the device in the method of inverting diverticula 100.

As noted above, the various components of the device for inverting diverticulum 115 can be moveable relative to each other. As well, in order to maintain a minimal profile of the device for inverting diverticulum 115 prior to use of the device for inverting diverticulum 115 in the method of inverting diverticula 100, the closer 120 can be used to retain the basket 124 of the basket shaft 122. Similarly, to prevent the inadvertent deployment or interaction of the closure clip 140 with the surrounding environment, the sheath 126 can be disposed about the clip tube 128, tube stop 132, and the closure clip 140 retained in between. FIG. 1B illustrates the initial configuration of the device for inverting diverticulum 115 after the basket 124 is released from the closer 120. As described, the sheath 126 is disposed about the clip tube 128, tube stop 132, and the closure clip 140.

Once the device for inverting diverticulum 115 has been inserted into the body, the device can be used to treat a diverticulum. FIG. 1L illustrates a flow chart of the method of inverting diverticula 100. Each of the steps of the method of inverting diverticula 100 are illustrated in FIGS. 1C-K. In the method of inverting diverticula 100, the device for inverting diverticulum 115 is used to treat a diverticulum 110 that is located on the surface of the outer wall of colon 116

FIG. 1C illustrates step 101 in the method of inverting diverticula 100. As illustrated, the outer wall of colon 116 includes a diverticulum 110 that protrudes from the diverticulum 110. In some embodiments, in step 101, the atraumatic tip 138 of the push rod 136 is extended to contact the top surface of the diverticulum 110. In some examples, the atraumatic tip 138 enters substantially perpendicular to the colon surface. Initial contact adjustments may be necessary in order to evenly contact the diverticulum 110.

Next, as illustrated in FIG. 1D, the diverticulum 110 is inverted at step 102. In some embodiments, as can be seen, the push rod 136 can extend distally such that the atraumatic tip 138 compresses the diverticulum 110 to force it to invert. As the diverticulum 110 inverts, the outer lip of the tissue of the diverticulum 110 can form an ostium that can provide the closure clip 140 with tissue to engage with.

Once the diverticulum 110 has been inverted, as illustrated in FIG. 1E, step 103 involves fully forming the inverted diverticulum 112 and preparing the basket 124 to engage with the surrounding tissue of the ostium 114. As can be seen, after the diverticulum 110 is inverted with the atraumatic tip 138 of the push rod 136, the sheath 126 with the retained clip tube 128, tube stop 132, and closure clip 140 can be further advanced into the inverted diverticulum 112. The advancing of the device into the inverted diverticulum 112 better allows the tines of the basket 124 to engage with the ostium tissue 114 in tension (not pictured in FIG. 1E). In some examples, prior to the basket 124 touching the tissue of the ostium 114, the diameter of the basket 124 can be adjusted to 2-3 mm greater than the ostium 114. In some embodiments, once the basket 124 engages with the ostium 114 tissue, the outer wall of colon 116 is depressed by approximately 1 inch.

Next, FIG. 1F illustrates step 104 which describes tissue acquisition by the basket 124 once the device for inverting diverticulum 115 is in position. As illustrated, while the tissue of the inverted diverticulum 112 and ostium 114 are still under tension by the inserted distal end of the device for inverting diverticulum 115, the basket 124 is closed about the ostium 114 to draw up the tissue evenly. In some embodiments, this is accomplished by advancing the closer 120 distally past the tines of the basket 124. As the closer 120 is advanced, the tines of the basket 124 are drawn together to capture the tissue of the ostium 114. In some embodiments, if the tines of the basket 124 slip or the creases of the captured tissue are largely asymmetrical, the basket 124 can be opened and step 104 can be repeated.

Once the tissue of the ostium 114 has been captured by the basket 124, step 105 as illustrated in FIGS. 1G-H disclose deploying and engaging of the closure clip 140 with the tissue of the ostium 114 while located in the inverted diverticulum 112. As illustrated in FIG. 1G, the inverted diverticulum 112 can be expanded (e.g. with gas) to allow the closure clip 140 to expand. As described above, the attached closure clip 140 can be expanded to allow the clip tines 142 to flare outwards by withdrawing the sheath 126 in a proximal direction. In some embodiments, the withdrawn sheath 126 allows the clip tines 142 of the closure clip 140 to flare outwards. In some embodiments, as illustrated in FIG. 1H, the clip tube 128 and the tube stop 132 are withdrawn proximally to engage with the tissue of the inverted diverticulum 112 and ostium 114. As discussed, in some examples, because the inverted diverticulum 112 is expanded with gas, this prevents the inverted diverticulum 112 tissue from bunching up over the tines. As illustrated, the clip tines 142 of the closure clip 140 are targeted to penetrate into the outer wall of colon 116.

In some examples, once the closure clip 140 has penetrated into the tissue, the method of inverting diverticula 100 can further include step 106 which illustrates the releasing of the closure clip 140 into the tissue. As illustrated in FIG. 1I, the inverted diverticulum 112 is longer inflated (e.g. the gas can be stopped). To release the closure clip 140 into the target tissue, the clip tube 128 can first be withdrawn proximally. In some examples, the proximal withdrawing of the locking structures 130 releases the closure clip 140 from the angle on the clip tube 128 and allows the closure clip 140 to begin to rotate into its final position. The tube stop 132 is not withdrawn so as to maintain the closure clip 140 on the device. Next, as illustrated in FIG. 1J, after the clip has rotated into its final position (e.g. planar position), the tube stop 132 is withdrawn in a proximal direction. In some examples, the tube stop 132 and the flange 134 slips through the center of the inserted closure clip 140. As well, as illustrated in FIG. 1J, the basket 124 can be opened to release the tissue of the ostium 114. In some embodiments (not pictured), the closure clip 140 can be sprung to the clip tube 128 such that the withdrawal of the tube stop 132 and flange 134 can be self-driven.

Finally, the device for inverting diverticulum 115 can be removed from the inverted diverticulum 112 in step 107 as illustrated in FIG. 1K. To remove the device for inverting diverticulum 115 from the inverted diverticulum 112, the push rod 136 and atraumatic tip 138 can be withdrawn gently to pull it past the implanted closure clip 140. As seen in FIG. 1K, once the push rod 136 and atraumatic tip 138 are removed from the inverted diverticulum 112, the closure clip 140 is allowed to rotate such that it is fully flattened. In some examples, as the closure clip 140 flattens, the clip tines 142 of the closure clip 140 capture the tissue of the ostium 114 so as to flatten and secure the inverted diverticulum 112 closed on the outer wall of colon 116.

FIGS. 2A-H illustrates another embodiment of the device for inverting diverticulum 215. FIG. 2A illustrates a side perspective of the distal end of the device for inverting diverticulum 215.

Turning now to another embodiment of a device for inverting diverticulum 215, in some embodiments the device for inverting diverticulum 215 is composed of a plurality of components that are disposed coaxially about each other. Similar to the method of inverting diverticula 100, in the method of inverting diverticula as illustrated in FIGS. 2B-H, each of the components of the device for inverting diverticulum 215 are configured to be moveable relative to each other.

In some embodiments, the device for inverting diverticulum 215 can include a closer 220, a basket shaft 222, a ramp tube 244, and a push rod 236. As can be seen, the device for inverting diverticulum 215 is largely similar to the device for inverting diverticulum 115 with a few adjustments. For example, in some embodiments, the device for inverting diverticulum 215 includes a ramp tube 244 that can further include a ramped portion 246 at the distal end of ramp tube 244. Unlike the closure clip 140 in the device for inverting diverticulum 115 that is attached to the locking structures 130 of the clip tube 128, the closure clip 240 is disposed about the push rod 236. In some embodiments, the atraumatic tip 238 of the device for inverting diverticulum 215 can further include an inner opening 237. Each of the tubular components of the device for inverting diverticulum 115 can be composed of hardened steel.

As illustrated in FIG. 2A, the push rod 236 is located at the center of the device for inverting diverticulum 215. In some embodiments, the push rod 236 includes an atraumatic tip 238 at the distal end. In some examples, at the connection point between the push rod 236 and the atraumatic tip 238, the outer surface of the push rod 236 and the inner surface of the atraumatic tip 238 can form an inner opening 237. In some examples, the inner opening 237 can be configured to accommodate a portion of the closure clip 240 to adjust the angle that the closure clip 240 is located on the push rod 236. In other embodiments, the inner opening 237 of the atraumatic tip 238 can help to keep the closure clip 240 in a “safe” position prior to delivery. As will be discussed below, when it is ready to deliver the closure clip 240, the atraumatic tip 238 can be withdrawn to guide the closure clip 240 into the ramped position.

In some examples, the ramp tube 244 is disposed coaxially about the push rod 236. In some variants, the ramp tube 244 includes a ramped portion 246 that is located at the distal end of the ramped portion 246. The ramp tube 244 can be moveable relative to the push rod 236. In some examples, the ramped portion 246 of the ramp tube 244 can move an attached closure clip 240 into the inner opening 237 of the atraumatic tip 238 to alter the angle of the closure clip 240. The ramped portion 246 can help to adjust the angle on which the closure clip 240 is retained on the push rod 236 as well as to adjust the angle that the closure clip 240 penetrates into the target diverticulum. The ramped portion 246 can flare the clip into delivery position.

In some variants, the basket shaft 222 can be disposed about the ramp tube 244. In some examples, the basket shaft 222 can include a basket 224 located at the distal end of the basket shaft 222. As the basket shaft 222 is configured to be moveable relative to the ramp tube 244, the basket shaft 222 can be extended or withdrawn proximal and/or distal to the other components of the device for inverting the diverticulum 215. As will be described in more detail below, the basket 224 can be configured to retain the tissue about the ostium of the inverted diverticulum. This can help to better deploy the closure clip 240 into the target tissue.

In some embodiments, the device for inverting diverticulum 215 can further include a closer 220. In some examples, the closer 220 is configured to be disposed about the internal components of the device for inverting diverticulum 215. As the closer 220 is moveable relative to the basket shaft 222, the closer 220 can extend distally to cover and retain the basket 224 of the basket shaft 222. In some examples, this can help to maintain the minimal profile of the device for inverting diverticulum 215 prior to use of the device in the method of inverting diverticula.

The device for inverting diverticulum 215 can be used to treat a diverticulum. FIGS. 2B-H illustrate one embodiment of the method of inverting diverticula. The method of inverting diverticula is generally similar to the method of inverting diverticula 100.

FIG. 2B illustrates step 201 in the method of inverting diverticula. As illustrated, in some embodiments, after the device for inverting diverticulum 215 is inserted through the trocar, the closer 220 can be withdrawn in a proximal direction to release the 22 and the basket 224. The device for inverting diverticulum 215 can then be advanced until the basket 224 is centered on the target diverticulum 210.

Next, at FIG. 2C, the method of inverting diverticula can include step 202 wherein the atraumatic tip 238 on the push rod 236 can be advanced in a distal direction to invert the diverticulum 210. In some embodiments, the distal end of the atraumatic tip 238 can be spring loaded to prevent excessive force from being placed on the diverticulum 210 and in order to accommodate diverticulum 210 of different sizes. In some examples, at step 202, the closure clip 240 is retracted into the inner opening 237 of the atraumatic tip 238. In this configuration, the clip tines 242 is in a “safe” position, such that the clip tines 242 do not interfere with any tissue as it is inserted into the inverted diverticulum 112. In some examples, the inverted diverticulum 212 can be inflated (e.g. with gas) to allow better presentation of the ostium 214 to the closure clip 240.

In some embodiments, the method of inverting diverticula can include step 203 illustrated in FIG. 2D. Here, the basket 224 of the basket shaft 222 is closed around the tissue of the ostium 214 formed from the inverted diverticulum 212 on the outer wall of colon 216. In some examples, the basket 224 can grab the tissue by advancing the closer 220 in a distal direction until it completely covers the length of the basket 224. This can allow the basket 224 to pull and secure the healthy tissue of the ostium 214 around the ramp tube 244.

As illustrated in FIG. 2E, the method of inverting diverticula can include step 204 wherein the closure clip 240 is ramped into a “deploy” position. In some examples, at step 204, the push rod 236 is withdrawn in a distal direction with respect to the ramp tube 244. The ramped portion 246 of the ramp tube 244 can guide the closure clip 240 into the “deploy” position. In some examples, in the “deploy” position, the clip tines 242 of the closure clip 240 are flared outwards and engage with the gathered tissue at the ostium 214 of the inverted diverticulum 212. In some embodiments (not illustrated) the closure clip 240 can be released by continuing to withdraw the atraumatic tip 238 in a proximal direction. While the inner ledge 237 holds the clip in place, the movement of the atraumatic tip 238 is under the clip and ejects the closure clip 240 into the tissue as the closure clip 240 is moved along the ramped portion 246 of the ramp tube 244. Once the closure clip 240 is free of the inner ledge 237, it can begin to expand outwardly to return to its original planar shape.

Once the clip tines 242 of the closure clip 240 has been inserted into the tissue of the ostium 214, the method of inverting diverticula can proceed to step 205. As illustrated in FIG. 2F, the closer 220 can be withdrawn in a distal direction. As the closer 220 is withdrawn, the basket 224 can be opened—thereby releasing the gathered tissue of the ostium 214 around the neck of the inverted diverticulum 212.

In some embodiments, the method of inverting diverticula can then include step 206 as illustrated in FIG. 2G. Once the clip tines 242 of the closure clip 240 are secured in the tissue of the ostium 214, the push rod 136 can be advanced in a distal direction. In doing so, the closure clip 240 is released from the inner opening 237 of the atraumatic tip 238. This can allow the closure clip 240 to rotate with the clip tines 242 in gripping tissue around the ramp tube 244.

Once the closure clip 240 has been released from the inner opening 237 of the atraumatic tip 238, the method of inverting diverticula can proceed to step 207 where the device for inverting diverticulum 215 is retracted from the inverted diverticulum 112. In some embodiments, once the closure clip 240 has been released, the ramp tube 244, the ramped portion 246 of the ramp tube 244, the push rod 236, and the atraumatic tip 238 of the push rod 236 can be retracted through the ostium 214 of the inverted diverticulum 112. In some examples, once the distal end of the device for inverting diverticulum 215 has been fully retracted, the closure clip 240 can closed to its natural flat shape and grip the healthy tissue of the ostium 214 together with the clip tines 242.

Finally, FIGS. 3A-S illustrate another embodiment of the device for inverting diverticulum 315. FIG. 3A illustrates a side perspective of the distal end of the device for inverting diverticulum 315. FIGS. 3B-L illustrate a plurality of views of the components in the device for inverting diverticulum 315. Many of the illustrations of the components of the device for inverting diverticulum 315 can be similarly applicable to the device for inverting diverticulum 115 and device for inverting diverticulum 215 discussed above.

The device for inverting diverticulum 315 has elements that resemble or are similar to the device for inverting diverticulum 115 and device for inverting diverticulum 215 described above. Accordingly, numerals used to identify features of the device for inverting diverticulum 115 and device for inverting diverticulum 215 are incremented by a factor of one hundred to identify like features of the device for inverting diverticulum 315. This numbering conventional generally applies to the remainder of the figures. Any component or step disclosed in any embodiment in this specification can be used in other embodiments.

In some embodiments, the device for inverting diverticulum 315 (as illustrated in FIG. 3A), can be composed of a plurality of components that are disposed coaxially about each other. As will be illustrated in the method of inverting diverticula, each of the components of the device for inverting diverticulum 315 are configured to be moveable relative to each other. Unlike the previous two embodiments, in some examples, the device for inverting diverticulum 315 does not include a basket for securing the ostium of the inverted diverticulum. As discussed above, in some embodiments, each of the tubular components of the device for inverting diverticulum 115 can be composed of hardened steel.

As seen in FIG. 3A, the push rod 336 is located at the center of the device for inverting diverticulum 315. As can be seen, in some embodiments, the closure clip 340 is disposed about the push rod 336 of the device. In some embodiments, the push rod 336 can include an atraumatic tip 338 at the distal end.

FIGS. 3B-D illustrate a plurality of views of the closure clip 340. Each of these illustrations can be applicable to any discussion of the closure clip provided above. FIG. 3B illustrates a top view of an embodiment of the closure clip 340 in its relaxed state. In its relaxed state, the closure clip 340 is flat with the clip tines 342 pointing inward. In some embodiments, this is the form the closure clip 340 will take after it has been delivered inside of the inverted diverticulum 112. In some embodiments, the closure clip 340 can have tines that are 1.88 mm. FIG. 3C illustrates the closure clip 340 as it is loaded on the locking structures 330 of the clip tube 328. In this loaded state, the closure clip 340 is in a semi-flared shape. Lastly, FIG. 3D illustrates the closure clip 340 as it is flared even more for delivery to capture the tissue in the ostium 314. As was discussed, and will be discussed below, in the various embodiments of the method of inverting diverticula, the flaring of the clip tines 342 provide the closure clip 340 with a broader reach to engage the surrounding tissue of the ostium 314.

In some examples, at the connection point between the push rod 336 and the atraumatic tip 338, the outer surface of the push rod 336 and the inner surface of the atraumatic tip 338 can form an inner opening 337. In some examples, the inner opening 337 can be configured to accommodate a portion of the closure clip 340 to adjust the angle that the closure clip 340 is located on the push rod 336. FIGS. 3G-I illustrate a side, top, and cross-sectional view of the atraumatic tip 338. As is illustrated, the inner opening 337 provided between the outer surface of the push rod 336 and the inner surface of the atraumatic tip 338 provides an opening to accommodate a portion of the closure clip 340. As well, as illustrated in FIG. 3H, in some embodiments, the inner opening 337 can include a ledge that is adjacent the outer surface of the push rod 336.

In some examples, a clip tube 328 can be disposed about the push rod 336. In some embodiments, the clip tube 328 includes locking structures 330 at the distal end of the clip tube 328. As will be discussed in more detail below, the locking structures 330 can engage with the closure clip 340 that is disposed about the push rod 336 to secure the closure clip 340 on the distal end of the device for inverting diverticulum 315. As noted above, because the clip tube 328 and the push rod 336 are moveable relative to each other, withdrawing or advancing the clip tube 328 can cause the locking structures 330 to interact with the closure clip 340 to alter the angle in which the clip tines 342 are flared on the distal end of the device.

FIGS. 3E-F illustrate the clip tube 328 and a bottom perspective view of the clip tube 328 with closure clip 340 engaged. As illustrated in FIG. 3E, the locking structures 330 can include a plurality of equally spaced structures. FIG. 3F illustrate the closure clip 340 as it is engaged with the locking structures 330. As seen, each apex of the closure clip 340 is linked around the locking structures 330 to create the loaded flare shape—wherein the clip tines 342 are flared outwards and pointed in a proximal direction.

FIGS. 3J-K provide an illustration of the interaction between the locking structures 330 of the clip tube 328, the closure clip 340, and the atraumatic tip 338 of the push rod 336. FIG. 3J illustrates the closure clip 340 as it is loaded on the clip tube 328. In some examples, as the atraumatic tip 338 is moved up towards the clip tube 328, the ledge in the inner opening 337 can bump the apices of the closure clip 340. This can push them upward to cause a larger flare. FIG. 3K illustrates the distal end of the device for inverting diverticulum 315 and the configuration of the closure clip 340 during delivery. The structure of the inner opening 337 and atraumatic tip 338 allows the closure clip 340 to be pulled into the tissue without being inverted due to the force.

Lastly, in some embodiments, the device for inverting diverticulum 315 can further include a sheath 326 that can be disposed about the clip tube 328. In some examples, as seen in FIG. 3L, the sheath 326 can be wide enough such that it can accommodate the clip tube 328 and the attached closure clip 340. In some examples, the purpose of the sheath 326 is to prevent the closure clip 340 or the locking structures 330 of the clip tube 328 from unintentionally interacting with any tissue prior to deployments of the closure clip 340. The sheath 326 therefore protects the surrounding tissue as the device for inverting diverticulum 315 is inserted. As will be seen, the sheath 326 can be removed prior to deployment.

As discussed above, the device for inverting diverticulum 315 can be used to treat a diverticulum. FIGS. 3M-S illustrate another embodiment of the method of inverting diverticula. However, as will be discussed below, unlike the method of inverting diverticula discussed above, the device for inverting diverticulum 315 in the method of inverting diverticula does not include a basket for capturing the tissue of an inverted diverticula.

FIG. 3M can illustrate step 301 in the method of inverting diverticula. As is illustrated, the outer wall of colon 316 can include a diverticulum 310 that protrudes from the surface of the colon. In some embodiments, the atraumatic tip 338 located at the distal end of the device for inverting diverticulum 315 can be used to manually invert the diverticulum 310.

FIG. 3N next illustrates step 302 which shows the distal end of the device for inverting diverticulum 315 located in the inverted diverticulum 312. The distal end of the atraumatic tip 338 can further push into the inverted diverticulum 312 such that the ostium 314 is disposed snugly about the sheath 326.

The method of inverting diverticula can then include step 303 as illustrated in FIG. 3O. In some embodiments, at step 303, the sheath 326 is withdrawn in a proximal direction such that the closure clip 340 is exposed within the inverted diverticulum 312. As discussed above, the closure clip 340 at this stage is in the configuration illustrated in FIG. 3C, wherein the closure clip 340 is in a semi-flared state. In some examples, the inverted diverticulum 312 can be inflated (e.g. with gas) to allow better presentation of the ostium 214 to the closure clip 340.

In some examples, as illustrated in FIG. 3P, the method of inverting diverticula can include step 304 wherein the push rod 336 and the atraumatic tip 338 is retracted in a distal direction. As the push rod 336 is retracted, the ledge located within the atraumatic tip 338 (not pictured) can pull the closure clip 340 in a proximal direction, causing the closure clip 340 to flare to a larger diameter. In some examples, the closure clip 340 can be in the configuration illustrated in FIG. 3D wherein the flaring of the clip tines 342 can provide the closure clip 340 with a broader reach in order to engage the surrounding tissue of the ostium 314.

Once the closure clip 340 is flared outwards, the method of inverting diverticula can then include step 305 wherein the entirety of the device for inverting diverticulum 315 is retracted in a proximal direction. In some embodiments, as illustrated in FIG. 3Q, the device for inverting diverticulum 315 can be retracted in a proximal direction, this can serve to seat the clip tines 342 of the closure clip 340 into the surrounding tissue of the ostium 314. In some examples, once the clip tines 342 are in the surrounding tissue of the ostium 314, the closure clip 340 is ready to be released.

In some examples, the method of inverting diverticula can then include step 306 in order to begin the step of releasing the closure clip 340 into the tissue of the ostium 314. As illustrated in FIG. 3R, the atraumatic tip 338 of the push rod 336 is advanced in a distal direction. This can relax the closure clip 340 from the flared configuration to a semi-flared configuration. In some examples, the advancing of the atraumatic tip 338 in the inverted diverticulum 112 can allow the closure clip 340 to be released from the locking structures 330 of the clip tube 328. In some embodiments, (not illustrated) the push rod 336 can include a ramped portion. The closure clip 340 can be released by continuing to withdraw the atraumatic tip 338 in a proximal direction to cause the closure clip 340 to be ejected from the push rod 336.

Once the closure clip 340 has been released from the locking structures 330 of the clip tube 328, the distal end of the device for inverting diverticulum 315 can be retracted from the inverted diverticulum 112. As illustrated in FIG. 3S, in some embodiments, the configuration of the closure clip 340 can allow the atraumatic tip 338 to be pulled through the center of the closure clip 340. As the distal end of the device for inverting diverticulum 315 is pulled from the ostium 314 of the inverted diverticulum 312, the closure clip 340 can close around the captured tissue of the ostium 314. In some examples, the closure clip 340 can take the form of the closure clip 340 illustrated in FIG. 3B.

FIG. 4A-B show side views of dimensional presentations of diverticula and their inverted counterparts. As can be seen in the dimensional presentations of diverticulum 400, there are a number of different presentations and geometries of diverticulum 410 in affected patients. Similarly, these same characteristics impact the resultant shape of the inverted diverticulum 412 and the ostium 414 once the diverticulum 410 is inverted. This is due to the underlying muscle fiber orientation and inversion techniques required to manage these varying diverticulum geometries. This can be seen in FIG. 4B that illustrates the dimensional presentations of inverted diverticulum 412 and the varying configurations of the ostium 414 for each of the inverted diverticulum. The variance in ostium 414 of inverted diverticulum can have a significant impact on the ability of the clips to engage the colonic tissue in order to penetrate the wall and close around healthy tissue and provide a complete seal. Further, at each of the steps for the method of inverting diverticula 100 discussed above, variations in anatomy and presentation of anatomy can significantly impact the success of a given step in the process and therefore compromise the overall success of the procedure.

A key factor in the success of treating diverticular disease is the effectiveness of the tissue gathering process. The surface of the serosa (outer portion of colonic tissue that must be engaged in order to gather issue) is very slick with an inherently firm characteristic to it. This makes it difficult to capture tissue in the grip of the tines of the tissue gathering basket portion of the device. However, this is a necessary step prior to clip placement. Existing devices fail to reliably engage tissue during the gathering process due to the slick nature of the serosal surface. Once captured by the grip of the tines of the basket, the tissue can be very difficult to retain within the tines of the basket throughout the clip placement process. Larger diverticulum and diverticulum located on curved portions of the colon often pose the most significant issues as additional tissue must be gathered in the tines in order to approximate the edges of the diverticulum ostium prior to clip placement. The tissue mass/volume tends to block the closure of the tines due to the folds of tissue getting trapped between the tines preventing closure and retention of tissue throughout the clip placement process. Further, the bunching of tissue provides an inconsistent surface topography for the clip tines to penetrate the healthy colonic tissue in order to approximate the edges and close the diverticulum. This then can lead to bunching of tissue between the clip tines and ultimately ineffective clip placement.

As well, as the diverticulum is inverted, the ostial shape turns more oval in the direction lying along the axis of the underlying muscle fibers in the muscularis layer of the colon. As a result, the basket portion of the tissue gathering device is not evenly placed around the rim of the ostium and will recruit tissue into the basket inconsistently leading to mis-orientation of clips relative to the ostium. Because of the uneven placement of the basket, as the basket closes, the tips of the tines of the basket often fail to engage and gain purchase on the slick and hard serosal colon tissue making tissue gathering inconsistent and challenging. Existing tissue gathering devices frequently have basket tines that do not point inward early enough in the tissue gathering process and therefore fail to gain purchase on the tissue. Additionally, the tips of these tines have single-point blunt elements that have proven ineffective in gaining an initial grip on the tissue even prior to attempts at closing the basket. Those same tine tips then do not have the opportunity to gain purchase on the tissue due to the incident angle of approach as described above.

In some embodiments, the gathering basket can include a space between the tines in order to collect and organize the tissue. To ensure that the tissue pleats and folds evenly between basket tines, the user can align and then push the basket over the inverted diverticulum into the bowel wall by 0.5-3.0 cm (depending on bowel insufflation). This places slight tension on the tissue to be gathered. This can predispose the folding up of the tissue between the tines as the basket is closed in tension. As well, this can relieve the tension on the tissue during basket close. In this way, the pleats can be more uniform between the basket tines than if the basket was just engaged on the surface without tension.

In other embodiments, instead of pushing the basket into the bowel, the basket can be rotated to pleat and organize the tissue. In some examples, anything from a 5 to 180 degree rotation can engage the tissue and naturally fold the tissue between basket tines as the tine pushes laterally on the tissue. The tension in the tissue is created by the tines pulling against the tissue as the tines are brought together as they close. The device can include a basket closer tube that can be advanced to secure tissue folded neatly between the tines. The rotate basket can be released during closing or thereafter. This method can work for any basket design that has an adequate tissue grip. As will be discussed below, the basket can include features on the distal sides of the basket tines to hold the tissue securely between the tines when rotating the basket and when the basket is fully closed.

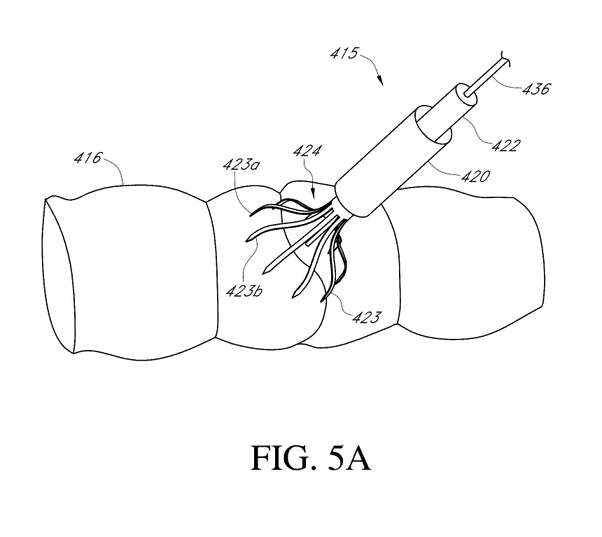

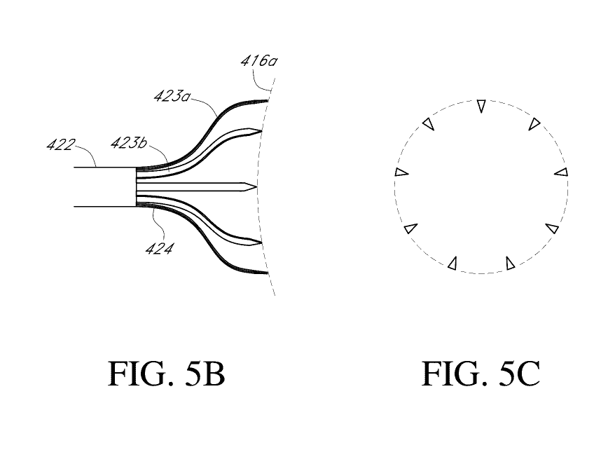

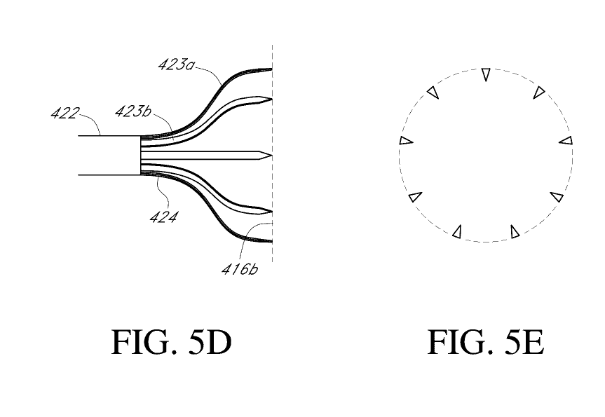

FIGS. 5-10 illustrate a plurality of embodiments of the gathering element. The gathering element can be composed of a variety of materials. In some embodiments, the gathering element is composed of an elastic metal such as nitinol, cobalt chromium, cobalt nitinol, etc. In other embodiments, the gathering element is composed of an elastic polymer.

FIGS. 5A-E show device for inverting diverticulum 415 with a basket 424 with non-uniform tines. The basket 424 of the device for inverting diverticulum 415 is directed to address the problem of an oval ostium by shaping the basket of the tissue gathering device in the shape of an oval. FIG. 5A shows the device for inverting diverticulum 415 in operation on an inverted diverticulum located on an outer wall of colon 416. The tissue gathering device of FIG. 5A includes a basket shaft 422, push rod 436, and a closer 420. The basket shaft 422 includes a basket 424 at the distal end. The basket 424 includes a plurality of basket tines 423. As illustrated, the basket tines 423 includes a plurality of first tine 423a that are longer and second tines 423b that are shorter. In some embodiments, there are an equal number of longer tines 423a and shorter tines 423b such that they are equally spaced in the basket 424. In some embodiments, the basket 424 includes equal numbers of longer tines 423a and shorter tines 423b that are adjacent. For example, the basket 424 can have two shorter tines 423b, two longer tines 423a, two shorter tines 423b, and two longer tines 423a. In other examples, the basket element 315 can have one shorter tine 423b, three longer tines 423a, one shorter tine 423b, and three longer tines 423a. In some examples, the groups of longer tines can have varying lengths such that the three longer tines 423a includes one longest tine that is spaced between two equal sized longer tines 423b. The basket shaft 422 is disposed about the push rod 436 (used to invert the diverticulum) and the closer 420 is disposed about the basket shaft 422. The basket shaft 422 and the closer 420 are movable relative to each other and the closer 420 can be moved distally to bring the longer tines 423a and shorter tines 423b of the basket 424 together.

In operation, once the diverticulum is inverted, the physician can rotate the basket 424 to align the oval shape of the ostium with the oval shape of the basket. Once the basket 424 is aligned with the oval shape of the ostium, the basket 424 can be more evenly engaged around the ostium and the tissue more evenly recruited in preparation for clip placement. FIGURE provides a side view of the basket 424 and FIG. 5E provides a view of the distal end of the different length tines of the basket 424 as it engages with the ostium. The different length tines of basket 424—the longer tines 423a and shorter tines 423b—engage with the tissue surrounding the ostium, and will form an oval on the surface of the outer wall of colon 416 that is seen in FIG. 5E. Such an oval orientation of basket tines will also serve to better engage tissue on the arched wall of colon 416a as seen in FIGS. 5B-C. In this case, the unequal lengths of the longer tines 423a and shorter tines 423b conform to the colon diameter for consistent contact and uniform tissue recruitment. In both cases, healthy colon tissue surrounding an ostium will be engaged more evenly and recruited into the tissue gathering basket in preparation for clip placement. In other embodiments, the distal ends of each of the longer tines 423a and shorter tines 423b can be modified with one of the embodiments described below.

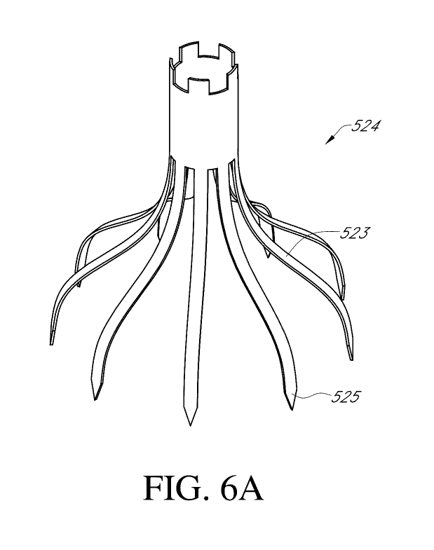

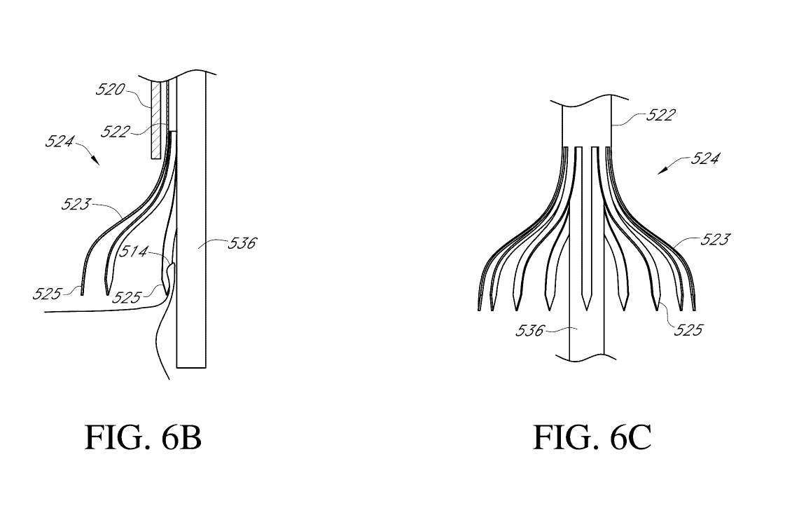

FIGS. 6A-C show a basket 524 with inward pointed tine ends, an embodiment of a tissue gathering device directed towards better engaging the tissue surrounding the ostium 514. The basket 524 with inward pointed tine ends includes a basket shaft 522 with a basket 524 located at the distal end. The basket 524 is composed of a plurality of basket tines 523 where the end of each of the basket tines 523 has a tine end 525 that is pointed inwardly. As shown, the tine tip modifications of the basket tines 523 have tines formed directionally away from the shaft of the device for most of the length of the basket tines 523. Towards the distal tip, the tines then curve back towards the shaft of the device to form a tine end 525 that is pointed inwardly.

In operation, as seen in FIG. 6B, the tissue gathering device includes the basket shaft 522 and a push rod 536 and a closer 520. The basket shaft 522 is disposed about the push rod 536 and the closer 520 is disposed about the basket shaft 522 such that the distal end of the closer 520 is proximal to the basket 524. The basket shaft 522 is positioned such that the basket tines 523 of the basket 524 are arranged about the ostium 514 such that the inward pointed tine end 525 of each of the basket tines 523 is engaged with the surrounding tissue of the ostium 514. The closer 520 is moved in a distal direction over the basket 524 such that the basket 524 is brought into a closed position as each of the basket tines 523 of the basket 524 are brought proximate to one another. As the basket tines 523 are brought proximal to each other, the inward pointed tine ends 525 are forced directionally into the ostium 514 early in the basket closure process. This drives the inward pointed tine end 525 of the basket tines 523 into the tissue as the basket is closing, ensuring that the incident angle of the basket tines 523 is such that they naturally gain purchase on the tissue of the ostium 514 as the basket 524 is closed.

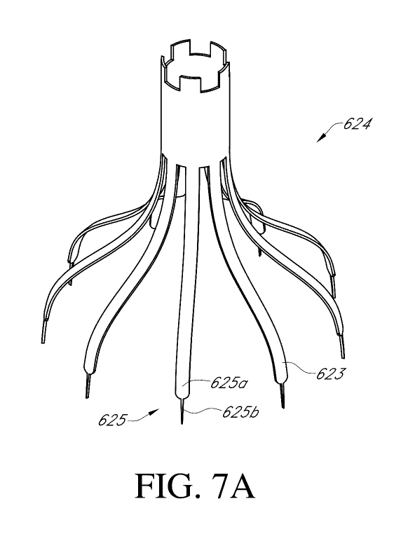

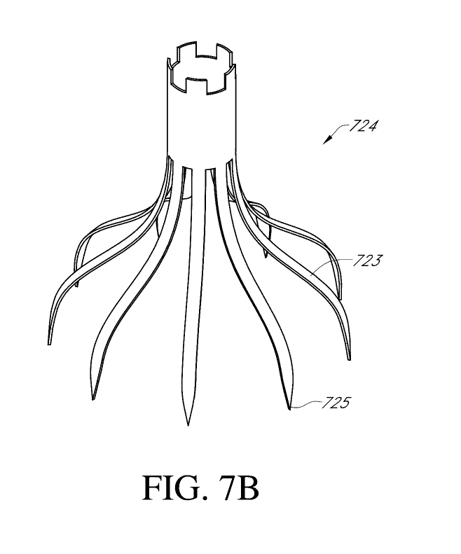

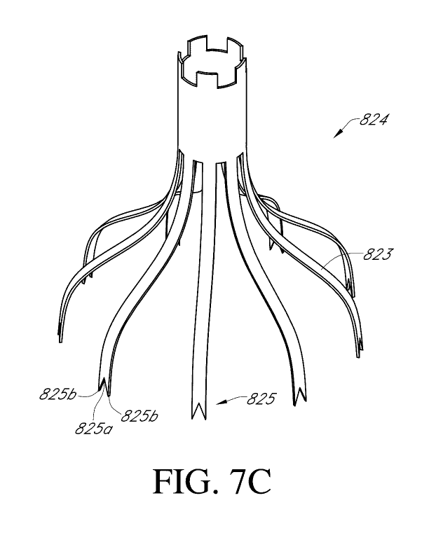

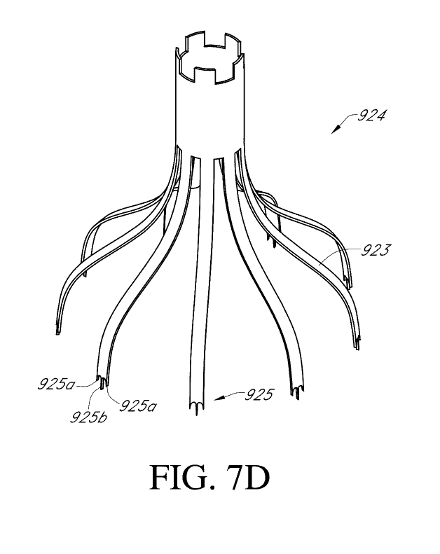

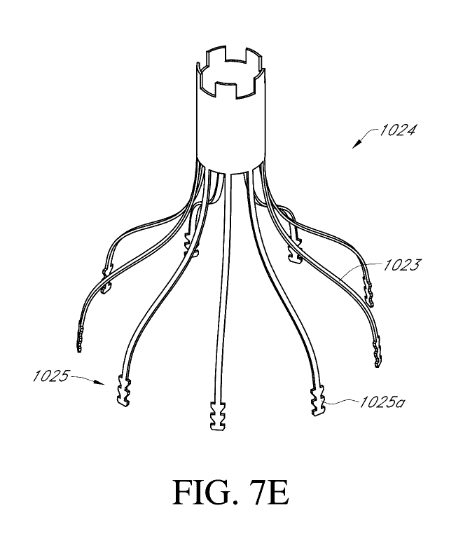

FIGS. 7A-E illustrate a variety of baskets with a different embodiments of tine ends. For example, FIG. 7A illustrates a basket 624 with a plurality of basket tines 623. Each of the tine ends 625 of the basket tines 623 includes an element 625a and an element 625b. As illustrated, in some embodiments, the element 625a can be wider and the element 625b can form a spike that protrudes from the distal end of the element 625a. In some examples, the element 625b that forms a spike can be 2 mm in length. FIG. 7B illustrates an embodiment of a basket 724 with tine ends 725 that forms a single point. In some embodiments, the basket 724 is composed of a plurality of basket tines 723 that has a tine end 725 that forms a single point. FIG. 7C illustrates an embodiment of a basket 824 with tine end 825 that includes two points. As illustrated, the basket 824 includes a plurality of basket tines 823 that has a tine end 825 that is formed from an element 825a and an element 825b. In some examples, the tine end 825 has element 825a that forms an arch at the distal end. The arched element 825a forms the two points of element 825b. FIG. 7D illustrates an embodiment of a basket 924 with tine end 925 formed of two points and a spike between the two points. In some examples, the basket 924 includes a plurality of basket tines 923. Each of the basket tines 923 includes a tine end 925 that has an element 925a and an element 925b. As illustrated, the element 925a forms two points on either side of the distal end of the tine end 925. The element 925b is a spike that is formed between the two points of element 925a. In some examples, element 925b can be approximately 1 mm. FIG. 7E illustrates an embodiment of a basket 1024 with tine end 1025 that have side clamping tines. As illustrated, the basket 1024 includes a plurality of basket tines 1023 with tine ends 1025 that are formed of a plurality of elements 1025a. In some examples, the elements 1025a of the tine end 1025 is formed of a plurality of shapes located along the distal end of the tine end 1025. The plurality of shapes can extend laterally past the width of each of the basket tines 1023. In some embodiments, as a result of the protruding shapes, the tine end 1025 can have side clamping properties.

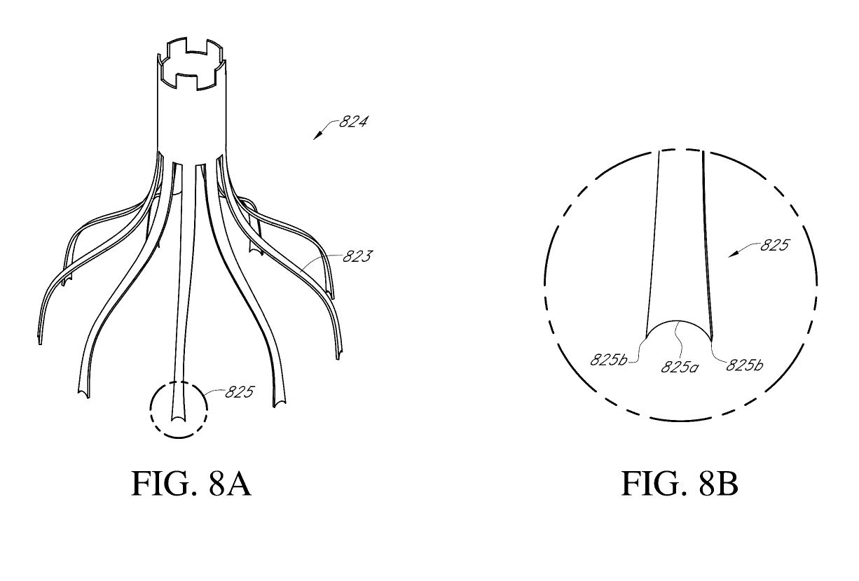

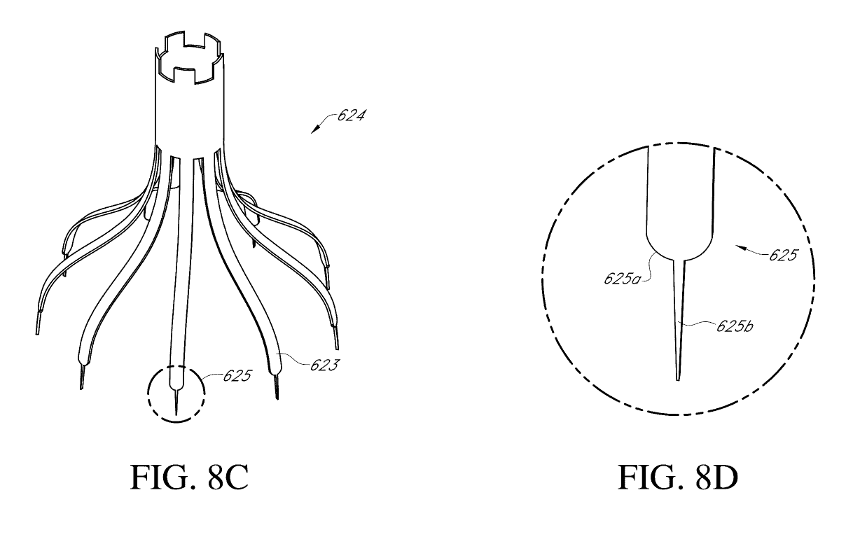

A detailed view of the embodiment of the tissue gathering device with modified basket tines of FIG. 7C is shown in FIGS. 8A-B. In some examples, the modified basket tines can improve the tissue captured between the tines. FIGS. 8A-B shows a basket 824 with double pointed tip tines. The basket 824 includes a plurality of basket tines 823 with a modified tine end 825 on the distal end of the basket tines 823. FIG. 8B provides a close up view of the tine end 825 of one of the basket tines 823. The tine end 825 includes an arch element 825a of the end of the tine end 825 that forms the distal end of the basket tines 823 into a pair of double point tips of element 825b. In operation, the tine tip modification of the distal end of basket tines 823 provides a wide base 825a and a pair of double point tips 825b in order to aid in tissue gathering and avoids the problem of single-point blunt elements as discussed above. The colon tissue is engaged through the double point tips 825b of the basket tines 823. The tine modifications of the basket tines 823 allow healthy colon tissue surrounding an ostium to be more easily engaged and recruited into the basket 824 in preparation for clip placement. In other embodiments, the distal end of the basket tines 823 can include more than one arch element 825a and will therefore form more than one point that will assist in tissue gathering.

FIGS. 8C-D is similar to FIGS. 8A-B with a different tine tip modification. FIG. 8C shows a tissue gathering device with a gathering element with sharp spike distal end as illustrated in FIG. 7A. The basket 624 includes a plurality of basket tines 623 with modified tine ends 625 on the distal end of the basket tines 623. FIG. 8D provides a close up view of the tine end 625 of one of the basket tines 623. The tine end 625 includes an element 625a that includes a flat end and an element 625b that forms a spike. As seen, the spike 625b can protrude from the distal end of the flat end 625a. In some embodiments, the length of the spike 625b provides sufficient pressure on the gathered tissue to gather colon tissue while having a low risk of piercing the colon tissue in an unhealthy way. In operation, the tine tip modification of the tine end 625 of basket tines 623 provide a wide base (the flat end 625a) and a spike 625b in order to aid in tissue gathering and avoid the problem of single-point blunt elements as discussed above. The colon tissue is engaged through the spike 625b of the basket tines 623. The tine modifications of the basket tines 623 allow healthy colon tissue surrounding an ostium to be more easily engaged and recruited into the basket 624 in preparation for clip placement. In other embodiments, the distal end of the basket tines 623 can include a plurality of spikes 625b attached to the end of flat end 625a. In other embodiments, the spike 625b can be attached at an angle such that the spike 625b if angled inwardly towards the center of the basket element 615.

Another difficulty faced by existing baskets of tissue gathering devices is the inability of the tines to close and retain tissue because of the uniform thickness/width of the tines of the basket. If too much tissue is gathered, or the tissue is too thick, the basket is unable to close to hold the tissue against the rest of the tissue gathering device. As a result, the tissue slips out and the tines of the inserted clip would be unable to engage consistently.

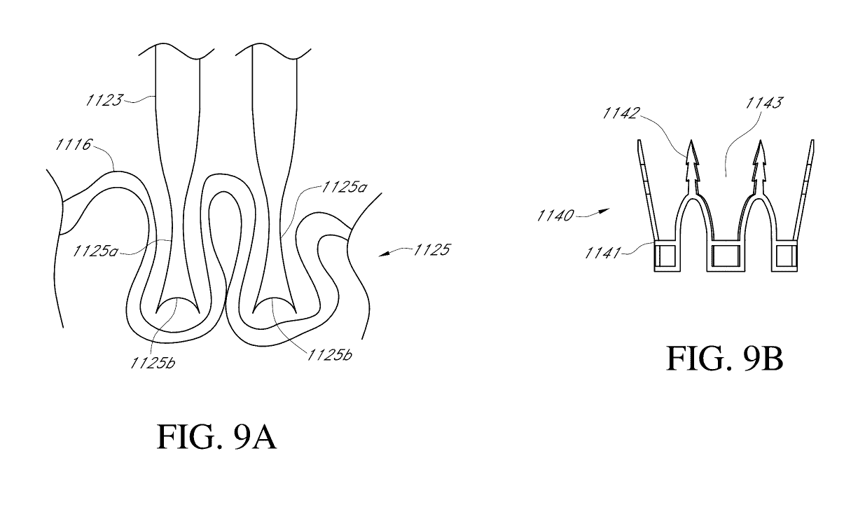

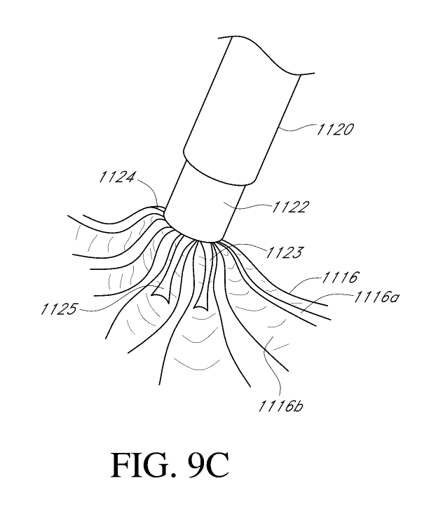

FIGS. 9A-C show a tissue gathering device with a gathering element with narrowed tine ends to address the uniform retention of tissue to facilitate clip placement. The gathering element with narrowed tine ends includes a basket shaft 1122 with a basket 1124 located at the distal end. The basket 1124 includes a plurality of basket tines 1123. FIG. 9A shows a close up of the basket tines 1123 as it is inserted about the diverticulum ostium. Each of the basket tines 1123 has an element 1125a that forms a narrowed waist 745. The tine end 1125 can also have an element 1125b that forms a flat distal end. FIG. 9B shows an inverted closure clip 1140 that is aligned to engage with the tissue gathered by the basket tines 1123. In operation, the basket 1124 of the basket shaft 1122 is aligned with the ostium of the inverted diverticulum located in the outer wall of the outer wall of colon 1116. The closer 1120 is disposed about the basket shaft 1122 is advanced in a distal direction and over the proximal end of the basket 1124. As the closer 1120 is advanced distally, the basket tines 1123 of the basket 1124 are brought in proximity with each other. As the basket tines 1123 are brought together, the tine end 1125 of each of the basket tines 1123 engages with the tissue of the ostium. The narrowed waist 1125a of each of the basket tines 1123 allow for more of the ostium tissue to be gathered as shown in FIG. 9A. The narrowed waist 1125 of the basket tines 1123 gathers ostium tissue such that the tissue of the outer wall of outer wall of colon 1116 form valleys 1116b and peaks 1116a about the ostium as seen in FIG. 9C. The tine end 1125 of the tine end 1125 push into the tissue of the ostium to form valleys 1116b where the tine ends 1125 contact the tissue of the outer wall of the outer wall of colon 1116. The tissue of the outer wall of colon 1116 form peaks 1116a between each of the tine ends 1125. The shape of the distal end of the tine end 1125 provides for consistent tissue capture and causes the tissue to be less prone to slipping out of the tine end 1125 during the placement of the closure clip 1140. The closure clip 1140 is composed of a plurality of petals 1141, each of the petals 1141 including a pair of clip tines 1142 and a petal body 1143. In one embodiment, there are as many clip tines 1142 as tine end 1125 of the basket 1124. The peaks 1116a and valleys 1116b formed from the tissue of the outer wall of colon 1116 allow the clip tines 1142 to have a better and more consistent landing zone during placement. The engagement of the closure clip 1140 is optimized through the placement of the clip tines 1142 in the valleys 1116b created by the tine end 1125 of the basket tines 1123. In one embodiment, the basket tines 1123 are further modified to include one or more of the modifications discussed above so as to further engage the tissue of the ostium.

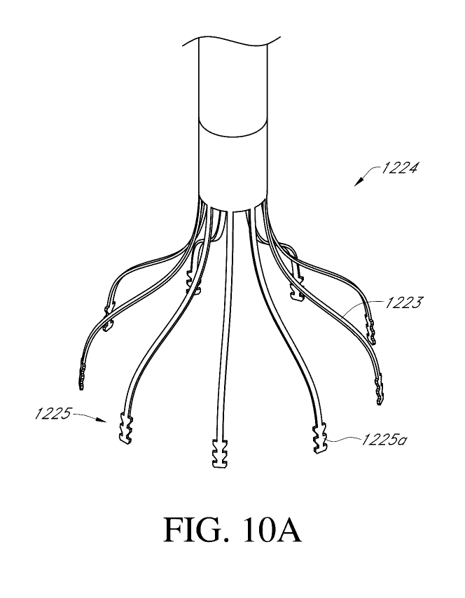

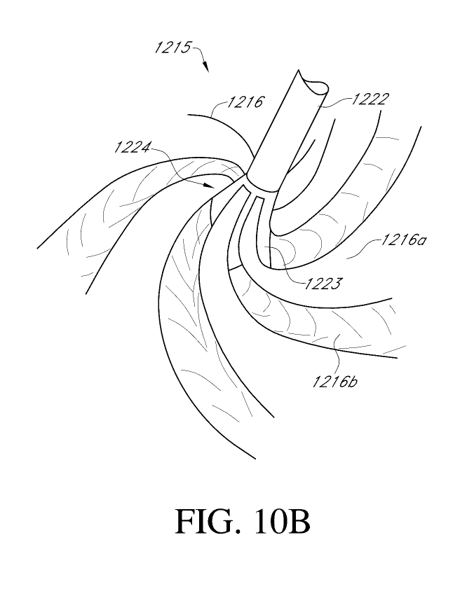

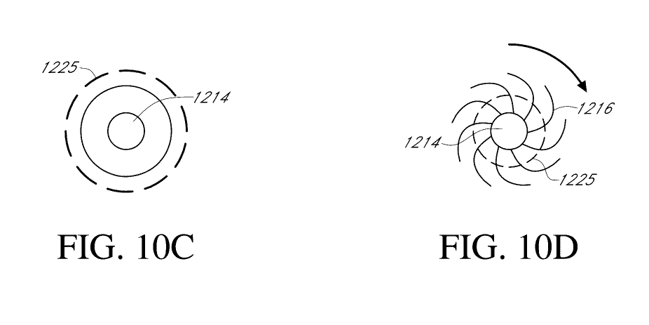

FIGS. 10A-D show another embodiment tissue gathering device with a gathering element with twisting basket to also enable uniform retention of tissue to facilitate clip placement. The gathering element with twisting basket is shown in FIG. 10A and includes a basket shaft 1222 with a basket 1224 located at the distal end. The basket 1224 includes a plurality of basket tines 1223, each of the basket tines 1223 including a basket shaft 1222 at the distal end. In operation, the basket 1224 of the basket shaft 1222 is aligned with the ostium 1214 of the inverted diverticulum that is located in the outer wall of colon 1216. The closure 1220 disposed about the basket shaft 1222 is advanced in a distal direction and over the proximal end of the basket 1224. As the closure 1220 is advanced distally, the basket tines 1223 of the basket 1224 are brought in proximity with each other. The basket 1224 can then be rotated once the tissue of the ostium 1214 is engaged. In some embodiments, the basket 1224 can rotate between 0-20 degrees, 20-40 degrees, or 40-60 degrees. In one embodiment, such rotation is accomplished through a handle, trigger or dial controlled by the operator. In another embodiment, the rotation can be controlled semi-automatically as the tissue of the ostium 1214 is gathered by the distal motion of the closure 1220 which brings the basket tines 1223 of the basket 1224 together. The gathering of the tissue is further facilitated by the grip elements 1125a located at the distal ends of each of the basket tines 1223. In some embodiments, the grip elements 1125a are protruding structures that prevent tissue from slipping. The grip elements 1125a increase the ability of the basket 1224 to capture and retain the tissue about the ostium 1214 during the rotation process by, in some embodiments, increasing the friction between the grip elements 1125a and the captured tissue. The grip elements 1125a ensures a grasp of the target tissue about the ostium 1214 and capture of the tissue of the ostium 1214 throughout the tissue gathering process in preparation for the clip placement. As well, similar to the embodiment in FIGS. 9A-C, peaks 1216a and valleys 1216b are formed in the outer wall of colon 1216—this time through the rotation of the basket 1224. The valleys 1216b are formed where the distal end of the basket tines 1223 engage with the tissue of the outer wall of colon 1216 and the peaks 1216a are formed as the basket tines 1223 of the basket 1224 are brought in proximity to each other. As discussed above, the formation of the valleys 1216b provide for better engagement of the tines of the clip as it is deployed. The rotated basket 1224 is released during or after the basket is closed by the closure 1220. FIGS. 10C-D show a cross-sectional view of the distal end of the basket 1224 as it interacts with the ostium 1214. FIG. 10C shows a view of the tine end 1225 of the basket tines 1223 as it engages with the tissue about the ostium 1214. FIG. 10D shows a view of the folds of the tissue of the outer wall of colon 1216 as the basket 1224 is twisted. As can be seen, while the twisting of the basket 1224 causes the basket tines 1223 to twist and create peaks 1216a and valleys 1216b in the outer wall of colon 1216, the tine end 1225 of the basket tines 1223 remains in place. Because the tine end 1225 of the basket 1224 remain in place as the basket is twisted (in this case in a clockwise direction), the location of the peaks 1216a and valleys 1216b of the outer wall of colon 1216 is more predictable.

The twisting configuration described above in FIGS. 10A-D can be included in any of the aforementioned embodiments. As described, by twisting the tines of the basket elements against the ostium, valleys and peaks of the tissue can form which provides better engagement for an optional clip to be attached.

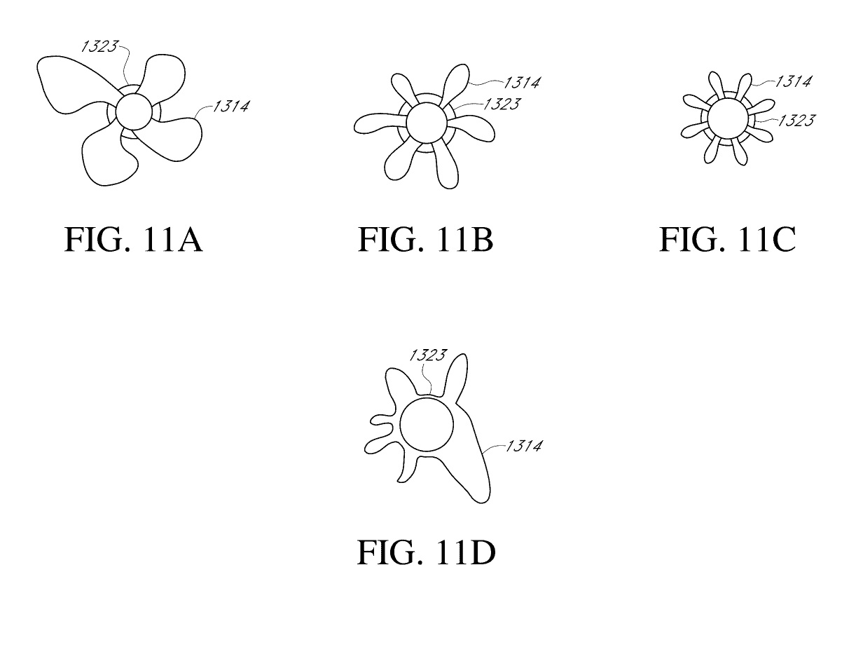

Finally, FIGS. 11A-D show a cross-sectional view of the distal end of the basket of the tissue gathering device about the ostium 1314 with a variable number of tines. As can be seen, there is increased variability in the tissue gathered and the ability of clips to engage that tissue if the number of tines of the basket is too low. If there are insufficient tines, the tissue tends to loop out of the basket in unpredictable ways, making it very difficult to target the clips to engage the captured tissue. As can be seen in FIGS. 11A-C, as the number of basket tines 1323 is increased from four, to six, to eight, the lobes of tissue about the ostium 1314 becomes increasingly uniform in shape between each of the basket tines 1323. The larger the loops of the tissue formed, the more difficult it is for the basket to close and for the tines of the clip to engage. As well, as seen in FIG. 11D a basket with too many or too wide of basket tines 1323 will cause the lobes of the tissue to slip out from between the basket tines 1323. In some embodiments, the basket will have 4, 5, 6, 7, or 8 tines.

While the description generally refers to colonoscopes and treatments within a colon, the devices and methods described herein are not limited to applications within a colon. They can be used to invert and/or treat outpocketings (e.g., diverticula, aneurisms, etc.) in any body lumen. Any reference to a colonoscope should be understood to be applicable to endoscopes generally, and similarly, any reference to a colon should be understood to be applicable to any body lumen.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A tissue gathering device, comprising:
a basket comprising a plurality of tines; and
a cylindrical shaft comprising a lumen, wherein the cylindrical shaft is disposed about a portion of the proximal end of the basket.

2. The device of claim 1, wherein the material of the basket is selected from an elastic polymer such as polyurethane, polyethylene terephthalate, or polyethyleneoxide or metal such as nitinol, cobalt-chromium.

3. The device of claim 1 wherein the basket comprises at least about 4, 6, 7 or 8 tines.

4. The device of claim 1, wherein the plurality of tines are of different lengths.

5. The device of claim 1, wherein the plurality of tines form an oval when the plurality of tines of the basket is depressed onto a surface.

6. The device of claim 1, wherein the plurality of tines are comprised of a first bend and a second bend, wherein the first bend extends the tine radially outward, and the second bend extends the tine radially inward along the length of the tine.

7. The device of claim 6, wherein the second bend has an angle of between about 0-180 degrees.

8. The device of claim 6, wherein the second bend has an angle of between about 0-20, 20-40, 40-60, 60-80, 80-100, 100-120, 120-140, 140-160 or 160-180 degrees.

9. The device of claim 1, wherein each of the plurality of tines has a diameter that widens along the length of each of the plurality of tines, and wherein each of the plurality of tines has a distal end with a recessed arch such that the distal end of each of the plurality of tines is configured to form a pair of spikes.

10. The device of claim 9, wherein the distal end of each of the plurality of tines has more than one recessed arch and forms a plurality of spikes.

11. The device of claim 1, wherein each of the plurality of tines has a diameter that widens along the length of each of the plurality of tines and ends in a wide base, wherein each of the plurality of tines has a narrow protrusion extending from the wide base.

12. The device of claim 9, wherein the distal end of each of the plurality of tines has more than one narrow protrusion.

13. The device of claim 1, wherein each of the plurality of tines has a variable diameter along the length of each of the plurality of tines that decreases and then increases so as to form a narrowed waist in each of the plurality of tines.

14. The device of claim 1, wherein each of the plurality of tines further comprises a shape element attached to the proximal end.

15. The device of claim 14, wherein the shape element is a bump or a protrusion.

16. The device of claim 1, further comprising a rotation mechanism, wherein the rotation mechanism is attached to the proximal end of the basket to allow rotation of the basket and the plurality of tines.

17. The device of claim 16, wherein the rotation mechanism is configured to rotate the basket and the plurality of tines between about 0-20, 20-40 or 40-60 degrees along an arc.

18. The device of claim 16, wherein the rotation mechanism is selected from a trigger, a dial or a lever.

19. The device of claim 1, wherein the plurality of tines form an oval.

20. A method for gathering tissue in treating a diverticulum, the method comprising:
positioning a distal end of a device along an outer wall of a colon at a diverticulum, wherein the device comprises a basket, a cylindrical shaft, and a pushing apparatus disposed coaxially between the basket and the cylindrical shaft, and wherein the distal end of the cylindrical shaft is disposed about the proximal end of the basket, and wherein the basket comprises a plurality of tines;
inverting the diverticulum by inserting the distal end of the pushing apparatus into the inverted diverticulum;
aligning the basket of the device with the inverted diverticulum such that the plurality of tines of the basket is disposed about the rim of the inverted diverticulum;
moving the cylindrical shaft in a proximal direction to retract the basket within the distal end of the cylindrical shaft; and
engaging the plurality of tines of the basket with the tissue around the inverted diverticulum.

21. The method of claim 20, wherein each of the tines has a first bend and second bend, and wherein the first bend extends the tine radially outward and the second bend extends the tine radially inward along the length of the tine.

22. The method of claim 21, further comprising the step of moving the cylindrical shaft in a distal direction such that the length of the basket between the first bend and the second bend is retracted into the distal end of the cylindrical shaft such that the angle of the second bend gathers the tissue around the inverted diverticulum.

23. The method of claim 20, wherein each of the tines has a diameter that widens along the length of each of the plurality of tines and has a distal end with a recessed arch such that the distal end of each of the plurality of tines is configured to form a pair of spikes.

24. The method of claim 23, further comprising engaging the spikes and recessed arch of the distal end of the plurality of tines with the tissue around the inverted diverticulum.

25. The method of claim 20, wherein each of the tines has a diameter that widens along the length of each of the plurality of tines and ends in a wide base, and each of the plurality of tines has a narrow protrusion extending from the wide base.

26. The method of claim 25, further comprising puncturing the tissue around the inverted diverticulum with the narrow protrusion from each of the plurality of tines, wherein the wide base limits the puncturing of the tissue by the narrow protrusion and aids in securing tissue about the diverticulum as the cylindrical shaft is moved in a distal direction.

27. The method of claim 20, wherein the device further comprises a clip comprising a plurality of clip tines configured to engage with each of the plurality of tines of the basket, and wherein the method further comprises engaging the plurality of tines with the plurality of clip tines of the clip such that the tissue about the diverticulum is secured between the plurality of tines and the plurality of clip tines.

28. A system for gathering tissue for use in treating a diverticulum, the system comprising:
a device comprising a basket comprising a plurality of tines;
a cylindrical shaft comprising a lumen, wherein the cylindrical shaft is disposed about a portion of the proximal end of the basket and wherein the lumen has a circular cross-section; and
a clip comprising a plurality of clip tines configured to engage with each of the plurality of tines of the basket.
